(19) United States
(12) Patent Application Publication
(10) Pub. No.: US 2025/0144008 A1
(43) Pub. Date: May 8, 2025

(54) COMPOSITIONS AND METHODS FOR TREATING SKIN DISORDERS

(71) Applicants: Vishagan Vanangamudi Sulur, Chennai (IN); Vanangamudi Subramaniam Sulur, Chennai (IN); Ramanathan Narayanan Lakshmi, Tamil Nadu (IN)

(72) Inventors: Vishagan Vanangamudi Sulur, Chennai (IN); Vanangamudi Subramaniam Sulur, Chennai (IN); Ramanathan Narayanan Lakshmi, Tamil Nadu (IN)

(21) Appl. No.: 18/940,649

(22) Filed: Nov. 7, 2024

Related U.S. Application Data
(60) Provisional application No. 63/597,279, filed on Nov. 8, 2023.

(51) Int. Cl. A61K 9/00; A61K 9/06; A61K 31/56; A61K 47/10; A61K 47/36; A61K 47/42; A61K 47/44; A61P 17/00; A61P 17/02

(52) U.S. Cl. CPC A61K 9/0014; A61K 9/06; A61K 31/56; A61K 47/10; A61K 47/36; A61K 47/42; A61K 47/44; A61P 17/00; A61P 17/02

(57) ABSTRACT

Provided herein are compositions for topical application to a subject, for treating epidermolysis bullosa (EB). Also disclosed are methods for treating skin in a person suffering from EB. The methods comprise providing a composition comprising fusidic acid and one or more pharmaceutically acceptable excipients; and topically applying, or instructing to topically apply, the composition to skin of a person suffering from EB. In some embodiments, the composition is topically administered to treat a non-healing wound, chronic wound or lesional skin in a patient suffering from EB, to reduce the likelihood of skin infections, to reduce the likelihood of scar formation, to prevent or reduce the risk of a skin blister from becoming infected, to treat intact blisters on skin, to treat skin dysbiosis, to alter bacterial diversity of the skin microbiome, or to increase the quality of life in a subject suffering from EB.

### CROSS-REFERENCE TO RELATED APPLICATIONS

This application claims the benefit of U.S. Provisional Application No. 63/597,279, filed Nov. 8, 2023, incorporated herein by reference in its entirety.

### TECHNICAL FIELD

The subject matter described herein relates to compositions and to methods for treating disorders of the skin, such as a non-healing wound, a chronic wound, lesional skin, and epidermolysis bullosa, with the compositions.

### BACKGROUND

Epidermolysis bullosa (EB) is a group of genetic skin diseases that cause the skin to blister and erode very easily. There are four main types of EB, which are classified based on the depth or level of blister formation. These include epidermolysis bullosa simplex (EBS), junctional epidermolysis bullosa (JEB), dystrophic epidermolysis bullosa (DEB), and Kindler syndrome (KS). Epidermolysis bullosa acquisita (EBA), is also similar to those of four types. In people with EB, blisters form in response to minor injuries or friction, such as rubbing or scratching. Inherited EB is a rare disease with a prevalence in the United States of 8.2 per million live births. EB is due to a mutation in at least one of 16 different genes. Some types are autosomal dominant while others are autosomal recessive. In most cases, the onset of EB is at birth or shortly after. It is estimated that half a million people worldwide are affected by the disease, many of whom are children.

The disease severity can range from mild to fatal. Complications may include esophageal narrowing, and the need for amputations. A potentially dangerous complication of EB is squamous cell carcinoma (SCC), which can be invasive and can metastasize in patients with EB. There is no clear consensus regarding the best treatment strategies and there is no cure for the condition. Disease management involves mostly supportive wound care, pain control, controlling infections, nutritional support, and prevention and treatment of complications. Monitoring for complications with laboratory testing and imaging studies is also important, although the frequency of these tests will vary depending on the type of EB and severity in each person.

The foregoing examples of the related art and limitations related therewith are intended to be illustrative and not exclusive. Other limitations of the related art will become apparent to those of skill in the art upon a reading of the specification and a study of the drawings.

### BRIEF SUMMARY

The following aspects and embodiments thereof described and illustrated below are meant to be exemplary and illustrative, not limiting in scope.

In one aspect, provided herein is a method for treating skin in a person suffering from epidermolysis bullosa (EB), such a person suffering from EB with a non-healing wound, chronic wound or lesional skin. In one embodiment, the method comprises providing a composition comprising fusidic acid and one or more pharmaceutically acceptable excipients; and topically applying, or instructing to topically apply, the composition to skin of a person suffering from EB.

In some embodiments, the subject provided with the composition is topically administered in a method for reducing the likelihood of scar formation in skin of a person suffering from EB. In some embodiments, the composition is topically administered in a method for preventing or reducing risk of a skin blister from becoming infected or for treating intact blisters on skin of a person suffering from EB.

In another aspect, provided herein is a method for (i) treating skin dysbiosis or (ii) altering the bacterial diversity of skin microbiome in a person in need of treatment. In one embodiment, the method comprises providing a composition comprising fusidic acid and one or more pharmaceutically acceptable excipients; and topically applying the composition to skin of a person in need of treatment. In some embodiments, the person in need of treatment has EB.

In some embodiments, the topical application increases the abundance of bacterial species of the skin microbiome that facilitate wound healing relative to the abundance of the bacterial species prior to the topical application. In certain embodiments, the increase in abundance is achieved by reducing the abundance of bacterial species that inhibit wound healing. In some embodiments, the topical application improves dysbiosis as measured by reduction or attenuation of cutaneous manifestations of wounded skin. In other embodiments, the composition is topically administered in a method for reducing the likelihood of a skin infection in a person suffering from EB. In certain embodiments, the composition is topically administered in a method for reducing the likelihood of creating or developing a microbial infection. In some embodiments, the reduction is a reduction in Staphylococcus aureus mutants, including methicillin-resistant strains of S. aureus.

In another aspect, provided herein is a method for improving the quality of life in a subject suffering from EB. In one embodiment, the method comprises providing a composition comprising fusidic acid and one or more pharmaceutically acceptable excipients; and topically applying, or instructing to topically apply, the composition to skin of a subject suffering from EB, thereby resulting in an improved quality of life as measured by one or more of a scale or scoring system selected from (i) a physician global assessment of pain, (ii) an investigator's global assessment of pain, (iii) a subject's global assessment of pain, and (iv) a measurement tool for itch intensity.

In some embodiments, a person provided with the composition is topically administered or instructed to topically apply the composition at least once daily. In other embodiments, the composition is topically administered or instructed to topically apply the composition twice daily, three times daily, four times daily, or as recommended. In some embodiments, the person is topically administered or instructed to topically apply the composition to skin presenting with EB symptoms and/or to skin presenting with EB symptoms and skin peripheral thereto. In certain embodiments, the peripheral skin includes skin with no presentation of EB symptoms. In some embodiments, the person is topically administered the composition to skin with intact blisters. In other embodiments, the person is topically administered, or instructed to topically apply, the composition to skin with intact blisters and open wounds.

In some embodiments, the composition is topically administered to the skin of person suffering epidermolysis bullosa simplex. In some embodiments, the composition is topically administered to the skin of a person suffering from dystrophic epidermolysis bullosa or junctional epidermolysis bullosa.

In some embodiments, the topical administration achieves re-epithelialization of damaged skin at a rate faster than untreated, damaged skin. In some embodiments, the topical administration reduces scar formation at a treated skin site relative to an untreated skin site.

In some embodiments, the fusidic acid is in the form of particles with a size of between about 100-500 nm. In some embodiments, the fusidic acid particles are uniformly dispersed in the composition. In some embodiments, the fusidic acid particles are uniformly dispersed in a composition, whereby the composition is an oil-in-water emulsion, a water-in-oil emulsion, an ointment, a gel, or a foam. In some embodiments, the fusidic acid particles are uniformly dispersed in the oil phase of an emulsion or ointment. In some embodiments, the fusidic acid particles are uniformly dispersed in a solvent in the composition.

In some embodiments, the fusidic acid is formed in situ in the composition from sodium fusidate and an acidic component. In some embodiments, the acidic component is hydrochloric acid, sulfuric acid, nitric acid, lactic acid, or citric acid.

In some embodiments, a topical composition comprising fusidic acid further comprises a biopolymer. In one embodiment, the biopolymer is chitosan.

In an embodiment, a composition is provided that is comprised of fusidic acid or a salt thereof; a biopolymer in an amount of greater than 1% w/w and less than about 5% w/w; one or more hydrophilic solvents selected from glycerin, propylene glycol and both glycerin and propylene glycol, a vegetable oil; and collagen.

In one embodiment, the fusidic acid or a salt thereof is fusidic acid.

In one embodiment, the vegetable oil is a hydrogenated vegetable oil. In one embodiment, the hydrogenated vegetable oil is present in the composition in an amount of between about 0.5-5.0% w/w. In one embodiment, the hydrogenated vegetable oil is polyoxyl 40 hydrogenated castor oil. In one embodiment, the polyoxyl 40 hydrogenated castor oil is present in the composition in an amount of between about 0.5-5.0% w/w.

In one embodiment, the collagen is marine collagen or hydrolyzed marine collagen. In one embodiment, the marine collage or hydrolyzed marine collagen is present in the composition in an amount of between about 0.05-0.8% w/w.

In one embodiment, the composition further comprises an oil mixture. In one embodiment, the oil mixture is present in the composition in an amount between about 8-15% w/w. In one embodiment, the oil mixture comprises a mineral oil and white petrolatum. In one embodiment, the mineral oil to white petrolatum are present in the oil mixture in a ratio of between 1:1 to 0.5:1. In one embodiment, the composition comprises between about 2.0-8.0% w/w mineral oil.

In one embodiment, the composition further comprises an emulsifier comprised of a fatty alcohol and an ethoxylated fatty alcohol. In one embodiment, the fatty alcohol is cetostearyl alcohol, polyoxyl 20 cetostearyl ether, or a mixture of cetostearyl alcohol and polyoxyl 20 cetostearyl ether. In one embodiment, the fatty alcohol is cetostearyl alcohol and the composition comprises between about 5-9% w/w cetostearyl alcohol. In one embodiment, the fatty alcohol is polyoxyl 20 cetostearyl ether and the amount of polyoxyl 20 cetostearyl ether in the composition is between about about 0.5-5.0% w/w.

In one embodiment, a composition, comprising 2% w/w fusidic acid, 1.25% w/w chitosan, 7% w/w glycerin, 16% w/w propylene glycol, 2% w/w polyoxyl 40 hydrogenated castor oil, 5% w/w light mineral oil, 7.5% w/w white petrolatum, 0.2% w/w marine hydrolyzed collagen, 2% w/w polyoxyl 20 cetostearyl ether, and 7.5% cetostearyl alcohol is provided.

In one embodiment, the composition further comprises one or more of water, lactic acid, benzoic acid, butylated hydroxytoluene, edetate disodium, and/or dibasic sodium phosphate.

In one embodiment, the composition is translucent and/or semi-solid.

The composition described herein further includes one or more pharmaceutically acceptable excipients, in an embodiment. In some embodiments, the pharmaceutically acceptable excipients include a lipophilic solvent. In some embodiments, the lipophilic solvent is polyethylene glycol or hexylene glycol. In some embodiments, the pharmaceutically acceptable excipients include a hydrophilic solvent. In some embodiments, the hydrophilic solvent is propylene glycol, glycerol, polyethylene glycol 200, or polyethylene glycol 400.

In some embodiments, the pharmaceutically acceptable excipients include an emulsifier. In some embodiments, the emulsifier is cetostearyl alcohol, polyoxyl 20 cetostearyl ether, sorbitan monooleate, sorbitan monostearate, stearyl alcohol, or a combination thereof.

In some embodiments, the pharmaceutically acceptable excipients include a wax. In some embodiments, the wax is paraffin or white petrolatum. In certain embodiments, the paraffin is light liquid paraffin, soft yellow paraffin, or hard paraffin.

In some embodiments, the pharmaceutically acceptable excipients include a preservative. In some embodiments, the preservative is benzoic acid, potassium sorbate, sodium benzoate, benzyl alcohol, methyl paraben, propyl paraben, or a combination thereof.

In some embodiments, the pharmaceutically acceptable excipients include a buffer. In some embodiments, the buffer comprises disodium hydrogen ortho phosphate, sodium hydrogen ortho phosphate, dibasic sodium phosphate, or a combination thereof.

In some embodiments, the composition further comprises an antioxidant. In some embodiments, the antioxidant is butylated hydroxytoluene or butylated hydroxyanisole.

In some embodiments, the composition further comprises a chelating agent. In one embodiment, the chelating agent is edetate disodium.

In certain embodiments, the composition comprises fusidic acid particles with a size between about 100-500 nm, white petrolatum or cetostearyl alcohol as a wax, polyoxyl 20 cetostearyl ether or sorbitan monostearate as an emulsifier, chitosan as a biopolymer at a molecular weight between about 300-650 kDa, and lactic acid and/or nitric acid as an acidic component. In another embodiment, the composition further comprises propylene glycol as a hydrophilic solvent, dibasic sodium phosphate a buffer, edetate disodium as a chelating agent, butylated hydroxytoluene as an antioxidant, benzoic acid as a preservative, and water. In certain embodiments, the composition does not comprise a drug compound other than fusidic acid and/or a biopolymer, or where fusidic acid and/or the biopolymer are the sole therapeutically active agents in the composition.

In addition to the exemplary aspects and embodiments described above, further aspects and embodiments will become apparent by reference to the drawings and by study of the following descriptions.

Additional embodiments of the present methods described herein will be apparent from the following description, drawings, examples, and claims. As can be appreciated from the foregoing and following description, each and every feature described herein, and each and every combination of two or more of such features, is included within the scope of the present disclosure provided that the features included in such a combination are not mutually inconsistent. In addition, any feature or combination of features may be specifically excluded from any embodiment of the present disclosure. Additional aspects and advantages of the present disclosure are set forth in the following description and claims, particularly when considered in conjunction with the accompanying examples and drawings.

### BRIEF DESCRIPTION OF THE DRAWINGS

FIGS. 1A-1B are computer generated images of cryo-SEM analysis of a composition with fusidic acid after freeze fracture revealing a size of crystalline fusidic acid of about 200 nm (FIG. 1A) and of about 400 nm (FIG. 1B).

FIG. 2 is a computer-generated images of cryo-SEM analysis of a composition with fusidic acid after freeze fracture and excess sublimation of the water phase to show a cauliflower morphology of the oil phase in the water matrix of the composition.

FIG. 3 is an x-ray diffractogram of a composition with fusidic acid, showing a distinct peak at around 7° that corresponds to the fusidic acid crystals.

FIG. 4 is a graph of wound area, in mm², as a function of days in animals with a burn wound treated once daily with the fusidic acid composition of Example 1 (closed triangles), Fucidin™ (open triangles), T-Bact™ (inverted triangles) and SOFRAMYCIN® (diamonds); untreated wounds on control animals (Group 5) are indicated by circles.

FIGS. 5A-5E are bar graphs of wound area, in cm², for wounds in a porcine partial thickness model treated with the fusidic acid and chitosan composition of Example 1 (Composition 1), a cream with chitosan (composition 2), a cream with fusidic acid (composition 3) and placebo (composition 4), at study day zero (FIG. 5A), study day 7 (FIG. 5B), study day 14 (FIG. 5C), study day 21 (FIG. 5D), and study day 28 (FIG. 5E).

FIGS. 6A-6D are bar graphs of wound scarring score for wounds in a porcine partial thickness model treated with the fusidic acid and chitosan composition of Example 1 (Composition 1), a cream with chitosan (composition 2), a cream with fusidic acid (composition 3) and placebo (composition 4), at study day 7 (FIG. 6A), study day 14 (FIG. 6B), study day 21 (FIG. 6C), and study day 28 (FIG. 6D).

FIG. 7 is a graph of colony forming units (CFU) as a function of study day from swabs taken from wounds treated with the fusidic acid and chitosan composition of Example 1 (composition 1, closed circles), a cream with chitosan (composition 2, closed triangles), a cream with fusidic acid (composition 3, closed squares) and placebo (composition 4, open diamonds).

FIGS. 8A-8D are bar graphs of wound CD31score for wounds in a porcine partial thickness model treated with the fusidic acid and chitosan composition of Example 1 (Composition 1), a cream with chitosan (composition 2), a cream with fusidic acid (composition 3) and placebo (composition 4), at study day 7 (FIG. 8A), study day 14 (FIG. 8B), study day 21 (FIG. 8C), and study day 28 (FIG. 8D).

FIG. 8E is a graph of the CD31 scores as a function of study day for wounds treated with the fusidic acid and chitosan composition of Example 1 (composition 1, closed circles), a cream with chitosan (composition 2, closed triangles), a cream with fusidic acid (composition 3, closed squares) and placebo (composition 4, open diamonds).

FIGS. 9A-9D are bar graphs of wound VEGF score for wounds in a porcine partial thickness model treated with the fusidic acid and chitosan composition of Example 1 (Composition 1), a cream with chitosan (composition 2), a cream with fusidic acid (composition 3) and placebo (composition 4), at study day 7 (FIG. 9A), study day 14 (FIG. 9B), study day 21 (FIG. 9C), and study day 28 (FIG. 9D).

FIG. 9E is a graph of the VEGF scores as a function of study day for wounds treated with the fusidic acid and chitosan composition of Example 1 (composition 1, closed circles), a cream with chitosan (composition 2, closed triangles), a cream with fusidic acid (composition 3, closed squares) and placebo (composition 4, open diamonds).

FIGS. 10A-10D are bar graphs of hydroxyproline amount (mg/g) for wounds in a porcine partial thickness model treated with the fusidic acid and chitosan composition of Example 1 (Composition 1), a cream with chitosan (composition 2), a cream with fusidic acid (composition 3) and placebo (composition 4), at study day 7 (FIG. 10A), study day 14 (FIG. 10B), study day 21 (FIG. 10C), and study day 28 (FIG. 10D).

FIG. 10E is a graph of the hydroxyproline amount (mg/g) as a function of study day for wounds treated with the fusidic acid and chitosan composition of Example 1 (composition 1, closed circles), a cream with chitosan (composition 2, closed triangles), a cream with fusidic acid (composition 3, closed squares) and placebo (composition 4, open diamonds).

FIGS. 11A-11D are bar graphs of collagen count for wounds in a porcine partial thickness model treated with the fusidic acid and chitosan composition of Example 1 (composition 1), a cream with chitosan (composition 2), a cream with fusidic acid (composition 3) and placebo (composition 4), at study day 7 (FIG. 11A), study day 14 (FIG. 11B), study day 21 (FIG. 11C), and study day 28 (FIG. 11D).

FIGS. 12A-12D are bar graphs of histopathology score (using the scoring system of Table 9-3 in Example 9) for wounds in a porcine partial thickness model treated with the fusidic acid and chitosan composition of Example 1 (Composition 1), a cream with chitosan (composition 2), a cream with fusidic acid (composition 3) and placebo (composition 4), at study day 7 (FIG. 12A), study day 14 (FIG. 12B), study day 21 (FIG. 12C), and study day 28 (FIG. 12D).

### DETAILED DESCRIPTION

### I. Definitions

Various aspects now will be described more fully hereinafter. Such aspects may, however, be embodied in many different forms and should not be construed as limited to the embodiments set forth herein; rather, these embodiments are provided so that this disclosure will be thorough and complete, and will fully convey its scope to those skilled in the art.

The singular forms “a,” “an,” and “the” include plural referents unless the context clearly dictates otherwise. Thus, for example, reference to a “polymer” includes a single polymer as well as two or more of the same or different polymers, reference to an “excipient” includes a single excipient as well as two or more of the same or different excipients, and the like.

The word “about” when immediately preceding a numerical value means a range of plus or minus 10% of that value, e.g., “about 50” means 45 to 55, “about 25,000” means 22,500 to 27,500, etc., unless the context of the disclosure indicates otherwise, or is inconsistent with such an interpretation. For example, in a list of numerical values such as “about 49, about 50, about 55, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein.

“Lesional” skin refers to wounded skin as evidenced by one or more of areas of blistering, areas of chronic damage, nonprogressive atrophic scarring, pigmentary changes, and/or atrophic erythema and/or to non-wounded skin with inflammatory erythema and/or scaling.

The phrase “pharmaceutically acceptable” is employed herein to refer to those compounds, salts, compositions, dosage forms, etc., which are—within the scope of sound medical judgment—suitable for use in contact with the tissues of human beings and/or other mammals without excessive toxicity, irritation, allergic response, or other problem or complication, commensurate with a reasonable benefit/risk ratio. In some aspects, “pharmaceutically acceptable” means approved by a regulatory agency of the federal or a state government, or listed in the U.S. Pharmacopeia or other generally recognized pharmacopeia for use in mammals (e.g., animals), and more particularly, in humans.

The term “treating” is used herein, for instance, in reference to methods of treating a skin condition, and generally includes the administration of a compound or composition which reduces the frequency of, or delays the onset of, symptoms of the skin condition in a subject relative to a subject not receiving the compound or composition. This can include reversing, reducing, or arresting the symptoms, clinical signs, and underlying pathology of a condition in a manner to improve or stabilize a subject's condition.

The compositions of the present disclosure can comprise, consist essentially of, or consist of, the components disclosed.

Where a range of values is provided, it is intended that each intervening value between the upper and lower limit of that range and any other stated or intervening value in that stated range is encompassed within the disclosure. For example, if a range of 1 μm to 8 μm is stated, it is intended that 2 μm, 3 μm, 4 μm, 5 μm, 6 μm, and 7 μm are also explicitly disclosed, as well as the range of values greater than or equal to 1 μm and the range of values less than or equal to 8 μm.

All percentages, parts and ratios are based upon the total weight of the topical compositions and all measurements made are at about 25° C., unless otherwise specified.

By reserving the right to proviso out or exclude any individual members of any such group, including any sub-ranges or combinations of sub-ranges within the group, that can be claimed according to a range or in any similar manner, less than the full measure of this disclosure can be claimed for any reason. Further, by reserving the right to proviso out or exclude any individual substituents, analogs, compounds, ligands, structures, or groups thereof, or any members of a claimed group, less than the full measure of this disclosure can be claimed for any reason.

Throughout this disclosure, various patents, patent applications and publications are referenced. The disclosures of these patents, patent applications and publications in their entireties are incorporated into this disclosure by reference in order to more fully describe the state of the art as known to those skilled therein as of the date of this disclosure. This disclosure will govern in the instance that there is any inconsistency between the patents, patent applications and publications cited and this disclosure.

For convenience, certain terms employed in the specification, examples and claims are collected here. Unless defined otherwise, all technical and scientific terms used in this disclosure have the same meanings as commonly understood by one of ordinary skill in the art to which this disclosure belongs.

### II. Methods of Treatment

In one aspect, a method for treating skin in a person suffering from epidermolysis bullosa (EB) is provided. The human skin consists of two layers: an outermost layer called the epidermis and a layer underneath called the dermis. In individuals with healthy skin, there are protein anchors between these two layers that prevent them from moving independently from one another (shearing). In people with EB the two skin layers lack the protein anchors that hold them together, resulting in extremely fragile skin. Since there is currently no cure for EB, treatment of EB aims to prevent complications and ease the pain of the blisters with appropriate wound care. It is difficult for wounds to heal in EB patients, and chronic wounds are common. Medication options include those that can help control pain and itching, medications that address complications such as sepsis (e.g., antibacterial agents or antibiotics), and medications that reduce inflammation (e.g., corticosteroids).

In one aspect, provided herein is a method for treating skin in a person suffering from EB. In some embodiments, the person suffering from EB has a non-healing wound, a chronic wound or skin lesion. In one embodiment, the method comprises providing a composition comprising fusidic acid and one or more pharmaceutically acceptable excipients; and topically applying, or instructing to topically apply, the composition to skin of a person suffering from EB. In some embodiments, topical administration of the composition reduces the likelihood of scar formation in the skin of a person suffering from EB. In other embodiments, provided herein are methods for preventing or reducing risk of a skin blister from becoming infected and/or treating intact blisters on skin of a person suffering from EB. In some embodiments, topical administration of the compositions described herein prevents or treats one or more signs or symptoms of EB, including presence of fluid-filled blisters on the skin, especially on the hands and feet due to friction, deformity or loss of fingernails and toenails, skin thickening on the palms and the soles of the feet, scalp blistering, scarring, thin-appearing skin (atrophic scarring), as well as tiny white skin bumps or pimples (milia).

Because the skin is the outermost tissue of the human body, it constitutes a physical barrier acting as a first line of defense by opposing the penetration of external agents, such as pathogenic microorganisms, antigens, and toxic substances. The skin is colonized by various microorganisms, such as bacteria, fungi, viruses, yeasts, fungi and archaea that coexist in symbiosis, together forming a microbial biofilm that interacts with our immune system. These microorganisms are distributed in a complex and well-defined equilibrium. This set of microorganisms, living in symbiosis with the human body skin, is known as the skin microbiota or dermobiota. This microbial film, together with the innate human immunity, create an external film or barrier that helps keep the skin healthy and protect it from external agents, pollution, and exogenous problems. In cases where the microbiological flora of the skin is altered, there is a transition from a state of balance defined as eubiosis to a condition of imbalance, known as dysbiosis, which is characterized by an alteration in the diversity and population of the skin microbiota. In these cases, the individual's skin becomes more susceptible to the action of external agents and there is an increased incidence of inflammatory diseases and skin infections.

Therefore, in some embodiments, provided herein is a method for (i) treating skin dysbiosis or (ii) altering bacterial diversity of skin microbiome. In one embodiment, the method comprises providing a composition comprising fusidic acid and one or more pharmaceutically acceptable excipients; and topically applying the composition to skin of a person in need of treatment.

In some embodiments, the topical application increases the abundance of bacterial species of the skin microbiome that facilitate wound healing relative to the abundance of the bacterial species prior to the topical application. In certain embodiments, the increase in abundance is achieved by reducing the abundance of bacterial species that inhibit wound healing. In some embodiments, the topical application reverses dysbiosis as measured by reduction or attenuation of cutaneous manifestations of wounded skin.

In some embodiments, administration of the composition reduces the likelihood of a skin infection in a person suffering from EB. In certain embodiments, the topical application achieves a reduction in the likelihood of developing antibiotic resistant mutants. In other embodiments, the reduction is a reduction in Staphylococcus aureus mutants, including methicillin-resistant strains of S. aureus.

In another aspect, provided herein is a method for improving the quality of life in a subject suffering from EB. In one embodiment, the method comprises providing a composition comprising fusidic acid and one or more pharmaceutically acceptable excipients; and topically applying, or instructing to topically apply, the composition to skin of a subject suffering from EB, whereby said topically applying improves quality of life as measured by one or more of a scale or scoring system selected from (i) a physician global assessment of pain, (ii) an investigator's global assessment of pain, (iii) a subject's global assessment of pain, and (iv) a measurement tool for itch intensity.

In some embodiments, the person topically administers, or is instructed to topically administer, the composition at least once daily, twice daily, three times daily, four times daily, or as recommended. In some embodiments, the person topically administers, or is instructed to topically administer, the composition to skin presenting with EB symptoms and/or to skin presenting with EB symptoms and skin peripheral thereto. In certain embodiments, the treated peripheral skin includes skin with no presentation of EB symptoms. In some embodiments, the person topically administers or is instructed to topically administer the composition to skin with intact blisters. In other embodiments, the person topically administers or is instructed to topically administer the composition to skin with intact blisters and open wounds.

In some embodiments, the composition is topically administered to the skin of a person suffering epidermolysis bullosa simplex. In some embodiments, the composition is topically administered to the skin of a person suffering from dystrophic epidermolysis bullosa or junctional epidermolysis bullosa.

In some embodiments, topical administration of the composition achieves re-epithelialization of damaged skin at a rate faster than untreated, damaged skin. In some embodiments, topical administration of the composition reduces scar formation at a treated skin site relative to an untreated skin site.

### III. Topical Compositions for Treatment

In some embodiments, the topical compositions for use in the methods disclosed herein are prepared in the form of a cream. In other embodiments, the topical compositions for use in the methods disclosed herein are prepared in the form of a gel. In some embodiments, the topical compositions disclosed herein include a single active agent. In some embodiments, the topical compositions disclosed herein include two or more active agents. In some embodiments, the compositions include an antibacterial agent or antibiotic agent, such as fusidic acid, a biopolymer such as chitosan, and/or a combination of the two. In some embodiments, the compositions further include one or more anti-inflammatory agents, such as corticosteroids and/or antioxidants.

In some embodiments, a topical composition comprises fusidic acid, an antibacterial agent. In some embodiments, the fusidic acid is in the form of particles or crystals with a size of between about 100-500 nm. In certain embodiments, the particle size of fusidic acid is between about 200-400 nm. In some embodiments, the particle size of fusidic acid is between about 120-200 nm. In some embodiments, the particle size is evaluated by scanning electron cryo-microscopy (Cryo-SEM). In other embodiments, the particle size is evaluated by x-ray diffraction.

In some embodiments, fusidic acid particles are uniformly dispersed in the composition. In some embodiments, the fusidic acid particles are uniformly dispersed in an oil-in-water emulsion, a water-in-oil emulsion, an ointment, a gel, or a foam. In some embodiments, the fusidic acid particles are uniformly dispersed in the oil phase of an emulsion or ointment. In some embodiments, the fusidic acid particles are uniformly dispersed in a solvent in the composition.

In some embodiments, the fusidic acid is formed in the composition in situ via reaction of sodium fusidate and an acidic component. In some embodiments, the acidic component is hydrochloric acid, sulfuric acid, nitric acid, lactic acid, or citric acid. In some embodiments, the acidic component is nitric acid. In some embodiments, the fusidic acid is the sole therapeutically active agent in the composition.

In some embodiments, a composition comprising fusidic acid further comprises a biopolymer, as described below. In some embodiments, the topical composition excludes an antibacterial agent, other than fusidic acid, or a biopolymer, other than chitosan.

The biopolymer provides, in some embodiments, a functional element of the compositions disclosed herein rather than acting as a mere carrier of other components in the composition. The biopolymer can serve as a base matrix for the cream formulations described herein. In some embodiments, the biopolymer is a naturally-occurring polymer or is a synthetic biopolymer prepared from a biological starting material. In one embodiment, the biopolymer is derived from a naturally-occurring biopolymer. In some embodiments, the naturally-occurring polymer is alginate, chitin, chitosan, a cellulose, a collagen, elastin, fibrin, gelatin, hyaluronic acid, keratin, a lectin, pectin, or starch.

In some embodiments, the biopolymer is therapeutically active. A biopolymer for use in the compositions and methods described herein may be film forming, mucoadhesive, non-toxic, biodegradable, and/or biocompatible with both healthy and infected skin, non-irritating to skin, possess antibacterial properties, possess viscosity-increasing properties, and/or possess wound healing properties. In some embodiments, the therapeutic activity of the biopolymer is evidenced by an ability to promote wound healing relative to a composition identical in all respects expect for presence of the biopolymer. In some embodiments, the biopolymer is the sole therapeutically active agent in the composition.

An exemplary biopolymer is chitosan. Chitosan is a linear polysaccharide composed of randomly distributed β-(1-4)-linked D-glucosamine (deacetylated unit) and N-acetyl-D-glucosamine (acetylated unit). It is known to have a number of commercial uses in agriculture and horticulture, water treatment, chemical industry, pharmaceuticals, and biomedicals.

Chitosan has natural anti-bacterial properties and helps actively heal and rejuvenate skin. Due to its unique physical properties, chitosan accelerates wound healing and wound repair. As a micro-film forming biomaterial, chitosan helps in reducing the width of a wound, controls oxygen permeability at the site, absorbs wound discharge, and is degraded by tissue enzymes. It is also useful in treating routine minor cuts and wounds, burns, keloids, diabetic ulcers and venous ulcers, is nonallergenic, non-toxic, biodegradable, non-irritating to skin, reduces itching by providing a soothing effect, acts as a moisturizer, and can be readily obtained from shrimps, squids and crabs.

Chitosan is positively charged, soluble in acidic to neutral solution, bioadhesive and readily binds to negatively charged surfaces such as mucosal membranes. In addition, chitosan enhances the transport of polar drugs across epithelial surfaces, facilitates rapid clot formation in blood, and has been approved for use in bandages and other hemostatic agents. Chitosan generally absorbs moisture from the atmosphere/environment and the amount absorbed depends upon the initial moisture content, temperature and relative humidity of the environment.

Chitosan is discussed in the US Pharmacopoeia forum with regard to its excipient category. The molecular weight of chitosan plays an important role in the topical composition. Higher molecular weight chitosan imparts a higher viscosity to the system and lower molecular weight chitosan imparts a lower viscosity to the system. When used in a cream, appropriate levels of viscosity are required to achieve good spreadability over the skin.

Since chitosan is a polymer, it is available in various grades depending upon the molecular weight. The various grades of chitosan include long chains, medium length chains & short length chain. The grades long, medium & short chain directly corresponds to the molecular weight of the chitosan. Generally, the long chain grade has a molecular weight in the range of 500-5,000 kDa, the medium chain grade has a molecular weight in the range of 1,000-2,000 kDa and the short chain grade has a molecular weight in the range of 50-1,000 kDa.

In some embodiments, the chitosan used in the topical composition described herein has a molecular weight ranging between 50-5000 kDa. In certain embodiments, the chitosan has a molecular weight between 300-650 kDa. In certain embodiments, the chitosan has a degree of deacetylation between 70-95%. In some embodiments, the topical composition comprises chitosan lactate, which is formed in the composition in situ via reaction of chitosan and lactic acid.

In some embodiments, a topical composition comprising a biopolymer (e.g., chitosan) further comprises an antimicrobial agent (e.g., fusidic acid). In some embodiments, the topical composition excludes an antibacterial, other than fusidic acid, or a biopolymer, other than chitosan.

In some embodiments, a topical composition comprising the biopolymer further comprises one or more antimicrobial agents. In some embodiments, the antimicrobial agent is an antibacterial agent. In some embodiments, the antibacterial agent is fusidic acid. In other embodiments, the antibacterial agent is fucidin, mupirocin, or framycetin sulfate. In yet other embodiments, the antibacterial agent is neomycin sulphate, sodium fusidate, calcium mupirocin, gentamycin, silver sulphadiazine, ciprofloxacin, framycetin sulfate, quiniodochlor, povidone-iodine, sisomicin, or nitrofural. Alternatively, or in addition, the antimicrobial agent is an antifungal agent. In some embodiments, the antifungal agent is nystatin, clotrimazole, econazole, ketoconazole, miconazole, sulconazole, oxiconazole, naftifine, terbinafine, miconazole nitrate, butenafine or griseofulvin.

In some embodiments, the antibacterial agent is present in the topical composition (e.g., cream or gel) at a concentration of 0.5-3% (w/w). In some embodiments, the antibacterial agent is present in the topical composition at a concentration of 1-2%.

The compositions described herein may further include one or more pharmaceutically acceptable excipients. In some embodiments, the pharmaceutically acceptable excipients include a lipophilic solvent. In some embodiments, the lipophilic solvent is polyethylene glycol or hexylene glycol. In some embodiments, the pharmaceutically acceptable excipients include a hydrophilic solvent. In some embodiments, the hydrophilic solvent is propylene glycol, glycerol, polyethylene glycol 200, or polyethylene glycol 400.

In some embodiments, the pharmaceutically acceptable excipients include an emulsifier. In some embodiments, the emulsifier is cetostearyl alcohol, polyoxyl 20 cetostearyl ether, sorbitan monooleate, sorbitan monostearate, stearyl alcohol, or a combination thereof. In some embodiments, the composition does not include polysorbates, such as polysorbate 80.

In some embodiments, the pharmaceutically acceptable excipients include white petroleum jelly. In some embodiments, the pharmaceutically acceptable excipients include a wax. In some embodiments, the wax is paraffin or white petrolatum. In certain embodiments, the paraffin is light liquid paraffin, soft yellow paraffin, or hard paraffin.

In some embodiments, the pharmaceutically acceptable excipients include a preservative. In some embodiments, the preservative is benzoic acid, potassium sorbate, sodium benzoate, benzyl alcohol, methyl paraben, propyl paraben, or a combination thereof.

In some embodiments, the pharmaceutically acceptable excipients include a buffer. In some embodiments, the buffer comprises disodium hydrogen ortho phosphate, sodium hydrogen ortho phosphate, or dibasic sodium phosphate.

In some embodiments, the composition further comprises an antioxidant. In some embodiments, the antioxidant is butylated hydroxytoluene or butylated hydroxyanisole.

In some embodiments, the composition further comprises a chelating agent. In one embodiment, the chelating agent is edetate disodium.

In an embodiment, the composition comprises fusidic acid in an amount from about 0.5-2.5% w/w, 0.75-2.5% w/w, 1.0-2.5% w/w, 1.25-2.5% w/w, 1.5-2.5% w/w, 1.75-2.5% w/w, 1.80-2.5% w/w, 1.90-2.5% w/w, 0.5-2.25% w/w, 0.75-2.25% w/w, 1.0-2.25% w/w, 1.25-2.25% w/w, 1.5-2.25% w/w, 1.75-2.25% w/w, 1.80-2.25% w/w, 1.90-2.25% w/w, 0.5-2.1% w/w, 0.75-2.1% w/w, 1.0-2.1% w/w, 1.25-2.1% w/w, 1.5-2.1% w/w, 1.75-2.1% w/w, 1.80-2.1% w/w, 1.90-2.1% w/w.

In an embodiment, the composition comprises a biopolymer in an amount of greater than about 1.0% w/w, 1.10% w/w, 1.11% w/w, 1.12% w/w, 1.13% w/w, 1.14% w/w, 1.15% w/w, 1.15% w/w, 1.17% w/w, 1.18% w/w, 1.19% w/w, 1.20% w/w, 1.21% w/w, 1.22% w/w, 1.23% w/w or 1.24% w/w and equal to or less than about 5.0% w/w, 4.0% w/w, 3.0% w/2, 2.5% w/w, 2.0% w/w, 1.9% w/w, 1.8% w/w, 1.75% w/w, 1.70% w/w, 1.65% w/w, 1.60% w/w, 1.55% w/w, 1.50% w/w, 1.45% w/w, 1.40% w/w, 1.35% w/w, or 1.30% w/w. In an embodiment, the biopolymer is present in the composition in an amount between about 1.1-5.0% w/w, 1.1-4.0% w/w, 1.1-3.0% w/w, 1.1-2.5% w/w, 1.1-2.0% w/w, 1.1-1.75% w/w, 1.1-1.50% w/w, 1.1-1.45% w/w, 1.1-1.40% w/w, 1.1-1.35% w/w, or 1.1-1.30% w/w. In an embodiment, the biopolymer is present in the composition in an amount between about 1.15-5.0% w/w, 1.15-4.0% w/w, 1.15-3.0% w/w, 1.15-2.5% w/w, 1.15-2.0% w/w, 1.15-1.75% w/w, 1.15-1.50% w/w, 1.15-1.45% w/w, 1.15-1.40% w/w, 1.15-1.35% w/w, or 1.15-1.30% w/w. In an embodiment, the biopolymer is present in the composition in an amount between about 1.20-5.0% w/w, 1.20-4.0% w/w, 1.20-3.0% w/w, 1.20-2.5% w/w, 1.20-2.0% w/w, 1.20-1.75% w/w, 1.20-1.50% w/w, 1.20-1.45% w/w, 1.20-1.40% w/w, 1.20-1.35% w/w, or 1.20-1.30% w/w. In an embodiment, the biopolymer is present in the composition in an amount of about 1.25% w/w. In an embodiment, the biopolymer is chitosan.

In an embodiment, the composition comprises a hydrophilic solvent other than or in addition to water, where the hydrophilic solvent is miscible with water. In an embodiment, the hydrophilic solvent is glycerin, polyethylene glycol, or propylene glycol, or a combination thereof. In an embodiment, the hydrophilic solvent is glycerin or propylene glycol or a combination of glycerin and propylene glycol. In an embodiment, glycerin is present in the composition in an amount between about 3-9% w/w, 3-8% w/w, 3-7.5% w/w, 4-9% w/w, 4-8% w/w, 4-7.5% w/w, 5-9% w/w, 5-8% w/w, 5-7.5% w/w, 6-9% w/w, 6-8% w/w, 6-7.5% w/w, 6.5-7.5% w/w. In an embodiment propylene glycol is present in the composition in an amount of less than 25% w/w. In an embodiment, propylene glycol is present in the composition in an amount between about 12-18% w/w, 13-18% w/w, 14-18% w/w, 15-18% w/w, 12-17% w/w, 13-17% w/w, 14-17% w/w, or 15-17% w/w. In an embodiment, the composition comprises a mixture of hydrophilic solvents where the mixture comprises at least two different hydrophilic solvents. In an embodiment, the mixture of hydrophilic solvents is present in the composition in an amount of less than about 25% w/w and greater than about 6% w/w. In an embodiment, the mixture of two different hydrophilic solvents is present in the composition at between about 6-24.5% w/w, 8-24.5% w/w, 9-24.5% w/w, 10-24.5% w/w, 12-24.5% w/w, 13-24.5% w/w, 14-24.5% w/w, 15-24.5% w/w, 16-24.5% w/w, 17-24.5% w/w, 18-24.5% w/w, 19-24.5% w/w, 20-24.5% w/w, 21-24.5% w/w or 22-24.5% w/w.

In an embodiment, the composition comprises a protein such as collagen, elastin or hyaluronic acid. In an embodiment, the protein is hydrolyzed. In an embodiment, the composition comprises collagen. In an embodiment, the collagen is bovine collagen, fowl collagen, marine collagen, or porcine collagen. In an embodiment, the collagen is hydrolyzed bovine collage, hydrolyzed marine collagen, or hydrolyzed porcine collagen. In an embodiment, the protein is present in an amount between about 0.05-0.8% w/w, 0.05-0.7% w/w, 0.05-0.6% w/w. 0.05-0.5% w/w, 0.05-0.4% w/w, 0.05-0.3% w/w, 0.05-0.25% w/w, 0.1-0.8% w/w, 0.1-0.7% w/w, 0.1-0.6% w/w. 0.1-0.5% w/w, 0.1-0.4% w/w, or 0.1-0.3% w/w, 0.1-0.25% w/w. In an embodiment, the composition comprises 0.2% w/w protein. In an embodiment, the composition comprises 0.2% w/w collagen. In an embodiment, the collagen is marine hydrolyzed collagen.

In an embodiment, the composition comprises a fully or partially hydrogenated vegetable oil. In an embodiment, the hydrogenated vegetable oil is castor oil, coconut oil, cottonseed oil, palm oil, sesame oil, soybean oil, or olive oil. In an embodiment, the hydrogenated vegetable oil is ethoxylated with ethylene oxide to form polyethylene glycol esters and/or ethers. In an embodiment, the hydrogenated vegetable oil is polyoxyl 40 hydrogenated castor oil. In an embodiment, the hydrogenated vegetable oil is present in the composition in an amount between about 0.5-5.0% w/w, 0.5-4.0% w/w, 0.5-3.5% w/w, 0.5-3.0% w/w, 0.5-2.5% w/w, 0.5-2.25% w/w, 1.0-5.0% w/w, 1.0-4.0% w/w, 1.0-3.5% w/w, 1.0-3.0% w/w, 1.0-2.5% w/w, 1.0-2.25% w/w, 1.5-5.0% w/w, 1.5-4.0% w/w, 1.5-3.5% w/w, 1.5-3.0% w/w, 1.5-2.5% w/w, 1.5-2.25% w/w, 1.75-5.0% w/w, 1.75-4.0% w/w, 1.75-3.5% w/w, 1.75-3.0% w/w, 1.75-2.5% w/w, or 1.75-2.25% w/w. In an embodiment, the composition comprises 2% w/w hydrogenated vegetable oil, such as polyoxyl 40 hydrogenated castor oil.

In an embodiment, the composition comprises an emulsifier system comprised of one or more of a vegetable oil, a hydrogenated vegetable oil, an ethoxylated hydrogenated vegetable oil, a fatty alcohol, and an ethoxylated fatty alcohol. In an embodiment, the composition comprises an emulsifier system comprised of two or more of or three of more of a vegetable oil, a hydrogenated vegetable oil, an ethoxylated hydrogenated vegetable oil, a fatty alcohol, and an ethoxylated fatty alcohol. In an embodiment, the fatty alcohol and/or the ethoxylated fatty alcohol is a C12-C22 alcohol, C12-C20 alcohol, C14-C22 alcohol, C14-C20 alcohol, C16-C22 alcohol, C16-C20 alcohol, or C16-C18 alcohol. In an embodiment, the fatty alcohol is a mixture of fatty alcohols with between 12-22 carbon atoms, 12-20 carbon atoms, 12-18 carbon atoms, 14-22 carbon atoms, 14-20 carbon atoms, 14-18 carbon atoms, 16-22 carbon atoms, 16-20 carbon atoms, or 16-18 carbon atoms. In an embodiment, the fatty alcohol is cetostearyl alcohol and in another embodiment the ethoxylated fatty alcohol is polyoxyl 20 cetostearyl ether. In an embodiment, the vegetable oil, hydrogenated vegetable oil, or ethoxylated hydrogenated vegetable oil are any of those disclosed herein. In an embodiment, the emulsifier system in the composition is present in an amount of between about 2-14% w/w, 3-14% w/w, 4-14% w/w, 5-14% w/w, 6-14% w/w, 7-14% w/w, 8-14% w/w, 9-14% w/w, 10-14% w/w, 11-14% w/w, 11-13% w/w or 11-12% w/w. In an embodiment, the composition comprises cetostearyl alcohol in an amount between about 5-9% w/w, 5.5-9% w/w, 5.5-8% w/w, 6-9% w/w, 6.5-9% w/w, 6.5-8% w/w, 7-9% w/w, 7-8.5% w/w, or 7-8% w/w. In an embodiment, the composition comprises the ethoxylated fatty alcohol polyoxyl 20 cetostearyl ether. In an embodiment, the ethoxylated fatty alcohol is present in the composition in an amount of between about 0.5-5.0% w/w, 0.5-4.0% w/w, 0.5-3.5% w/w, 0.5-3.0% w/w, 0.5-2.5% w/w, 0.5-2.25% w/w, 1.0-5.0% w/w, 1.0-4.0% w/w, 1.0-3.5% w/w, 1.0-3.0% w/w, 1.0-2.5% w/w, 1.0-2.25% w/w, 1.5-5.0% w/w, 1.5-4.0% w/w, 1.5-3.5% w/w, 1.5-3.0% w/w, 1.5-2.5% w/w, 1.5-2.25% w/w, 1.75-5.0% w/w, 1.75-4.0% w/w, 1.75-3.5% w/w, 1.75-3.0% w/w, 1.75-2.5% w/w, or 1.75-2.25% w/w. In an embodiment, the composition comprises 2% w/w ethoxylated fatty alcohol.

In an embodiment, the composition comprises a petroleum based or petroleum derived oil. In an embodiment, the petroleum based oil is mineral oil, and in an embodiment is light mineral oil. In an embodiment, the petroleum based oil is present in the composition in an amount between about 2.0-8.0% w/w, 2.0-7.5% w/w, 2.0-7.0% w/w, 2.0-6.5% w/w, 2.0-6.0% w/w, 2.0-5.5% w/w, 2.5-8.0% w/w, 2.5-7.5% w/w, 2.5-7.0% w/w, 2.5-6.5% w/w, 2.5-5.5% w/w, 2.5-5.5% w/w, 3.0-8.0% w/w, 3.0-7.5% w/w, 3.0-7.0% w/w, 3.0-6.5% w/w, 3.0-5.5% w/w, 3.0-5.5% w/w, 3.5-8.0% w/w, 3.5-7.5% w/w, 3.5-7.0% w/w, 3.5-6.5% w/w, 3.5-6.0% w/w, 3.5-5.5% w/w, 4.0-8.0% w/w, 4.0-7.5% w/w, 4.0-7.0% w/w, 4.0-6.5% w/w, 4.0-5.5% w/w, or 4.5-5.5% w/w. In an embodiment, the petroleum based oil is a liquid at room temperature (25° C.). In an embodiment, the petroleum based oil is present in the composition in an amount of about 5% w/w. In an embodiment, the petroleum based oil is light mineral oil.

In an embodiment, the composition comprises an oil mixture comprised of a mineral oil and white petrolatum. In an embodiment, the oil mixture is present in the composition in an amount between about 8-15% w/w, 9-15% w/w, 10-15% w/w, 8-14% w/w, 9-14% w/w, 10-14% w/w, 11-14% w/w, 8-13% w/w, 9-13% w/w, 10-13% w/w, 11-13% w/w or 12-13% w/w. In an embodiment, the oil mixture is comprised or consists of an oil that is a liquid at room temperature (about 25° C.) and an oil that is a solid or semi-solid at room temperature (about 25° C.). In embodiment, the oil that is liquid at room temperature is a mineral oil. In an embodiment, the oil that is a solid or a semi-solid at room temperature is white petrolatum. In an embodiment, the oil mixture comprises a ratio of liquid at room temperature oil to solid or semi-solid oil at room temperature of between about 1:1 to 0.5:1, 0.9:1 to 0.5:1, 0.8:1 to 0.5:1, 0.7:1 to 0.5:1 or 0.65:1 to 0.55:1.

In an embodiment, the composition excludes stearyl alcohol, sorbitan monostearate, or both stearyl alcohol and sorbitan monostearate.

In an embodiment, the composition is a transparent semi-solid at room-temperature (about 25° C.). In an embodiment, the composition is a transparent semi-solid when applied topically to a skin surface of a mammal, e.g., from about 32-35° C. or from about 32-36.9° C. In an embodiment, the transparent semi-solid is a gel. In an embodiment, the composition is clear, by which is meant an absence of appreciable cloudiness or haziness. In an embodiment, the composition is transparent, by which is meant light is able to pass without appreciable scattering. In an embodiment, the composition is characterized by non-Newtonian shear-thinning.

In some embodiments, the composition includes no active agents other than the antibacterial and/or the biopolymer. In some embodiments, the composition contains no active agents other than fusidic acid and/or chitosan.

In other embodiments, the composition further includes one or more additional active agents. In some embodiments, the addition active agents include one or more anti-inflammatory agents. In some embodiments, anti-inflammatory agent is a corticosteroid. Exemplary corticosteroids include clobetasol, betamethasone, mometasone, prednicarbate, methylprednisolone, triamcinolone, halobetasol, halcinonide, desoximetasone, fluocinonide, hydrocortisone, prednisolone, fluocortolone, chlorocortolone, fluocinolone, diflucortolone, desonide, dexamethasone, alclomethasone and desoximethasone, or any pharmaceutically acceptable salt or ester thereof, or mixture thereof.

In some embodiments, the anti-inflammatory agent is an antioxidant. In some embodiments, the antioxidant is butylated hydroxytoluene (BHT), butylated hydroxyanisole, tetrahexyldecyl ascorbate, methylgentisate, L-carnosine, tert-butylhydroquinone (TBHQ), and glutathione, including derivatives, combinations, and mixtures thereof. In some embodiments, the antioxidant is a vitamin, including free acids, derivatives, or precursors thereof. Exemplary vitamins include the above-described antioxidants, as well as lipophilic vitamins, such as tocopherol (i.e. vitamin E) and ascorbic acid (i.e. vitamin C), vitamin A, including free acids or derivatives thereof and precursors thereof, including retinoids, such as retinol, retinal, and retinyl esters, such as retinyl acetate, retinyl butyrate, retinyl propionate, retinyl octanoate, retinyl laurate, retinyl palmitate, retinyl oleate, and retinyl linoleate, tretinoin, isotretinoin, and bexarotene; and carotenoids; B-complex vitamins, including B1: thiamine, B2: riboflavin, B6: pyridoxin, panthenol, and pantothenic acid; vitamin B12 and combinations thereof; vitamin D, biotin, vitamin K, water-soluble derivatives thereof, and the like. In some embodiments, the composition is a cream. In other embodiments, the composition is an ointment. In another embodiment, the composition is a gel. Reference herein to a composition contemplates a composition in the form of a cream, ointment, paste or gel. The composition is suitable for topical application to skin.

In some embodiments, the composition is prepared to provide a viscosity ensuring that the active agents are distributed uniformly on an applied area for efficient drug release and diffusibility. In some embodiments, the viscosity is between 50,000-75,000 centipoise units (cPs).

In some embodiments, the composition is prepared to provide a shelf-life stability for at least 8 months, 12 months, 16 months, 20 months, 24 months, or more. As used herein, the term “shelf-life stability” refers to a composition comprising less than about 2-3% loss in bioactivity over a given time period. In embodiments, the bioactivity is with respect to fusidic acid. In embodiments, the bioactivity is with respect to chitosan.

In certain embodiments, the composition comprises fusidic acid in the form of particles with a size of between about 100-500 nm, a wax selected from white petrolatum and cetostearyl alcohol, an emulsifier selected from polyoxyl 20 cetostearyl ether and sorbitan monostearate, a biopolymer selected from chitosan at a molecular weight of between about 300-650 kDa and a degree of deacetylation between 70-95%, and an acidic component of lactic acid and/or nitric acid. In another embodiment, the composition further comprises one or more of a hydrophilic solvent comprising propylene glycol, a buffer of dibasic sodium phosphate, a chelating agent of edetate disodium, an antioxidant of butylated hydroxytoluene, a preservative of benzoic acid, and water. In an embodiment, the water is a solvent purified water. In other embodiments, the water is distilled water. In certain embodiments, the composition does not comprise a drug compound other than fusidic acid and/or chitosan, or where fusidic acid and/or chitosan are not the sole therapeutically active agents in the composition. In other embodiments, the composition excludes stearyl alcohol, sorbitan monostearate, or both stearyl alcohol and sorbitan monostearate.

In an embodiment, the composition comprises fusidic acid, a biopolymer, one or more hydrophilic solvents, a hydrogenated vegetable oil, an oil mixture, collagen, and/or an emulsifier comprised of a fatty alcohol and/or an ethoxylated fatty alcohol. In an embodiment, the composition is translucent and/or semi-solid; e.g., the composition is a gel. In an embodiment, the composition is a cream.

In an embodiment, the composition comprises fusidic acid, greater than 1% w/w or at least about 1.1% w/w of a biopolymer, one or more hydrophilic solvents, a hydrogenated vegetable oil, an oil mixture, collagen, and/or an emulsifier comprised of a fatty alcohol and/or an ethoxylated fatty alcohol. In an embodiment, the composition is translucent and/or semi-solid; e.g., the composition is a gel. In an embodiment, the composition is a cream. In an embodiment, the biopolymer is chitosan. In an embodiment, the amount of biopolymer in the composition is equal to or less than about 5.0% w/w. In an embodiment, the amount of biopolymer in the composition is in a range recited herein for this ingredient.

In an embodiment, the composition comprises fusidic acid, greater than 1% w/w or at least about 1.1% w/w of a biopolymer, one or more hydrophilic solvents selected from glycerin, propylene glycol and both glycerin and propylene glycol, a hydrogenated vegetable oil, an oil mixture, collagen, and/or an emulsifier comprised of a fatty alcohol and/or an ethoxylated fatty alcohol. In an embodiment, the biopolymer is chitosan. In an embodiment, the amount of biopolymer in the composition is equal to or less than about 5.0% w/w. In an embodiment, the amount of biopolymer in the composition is in a range recited herein for this ingredient. In an embodiment, the hydrophilic solvent is glycerin and propylene glycol. In an embodiment, the amount of each hydrophilic solvent is in a range recited herein for these solvents. In an embodiment, the composition is translucent and/or semi-solid; e.g., the composition is a gel. In an embodiment, the composition is a cream.

In an embodiment, the composition comprises fusidic acid, greater than 1% w/w or at least about 1.1% w/w of a biopolymer, one or more hydrophilic solvents selected from glycerin, propylene glycol and both glycerin and propylene glycol, a hydrogenated vegetable oil, an oil mixture, collagen, and/or an emulsifier comprised of a fatty alcohol and an ethoxylated fatty alcohol. In an embodiment, the composition is translucent and/or semi-solid; e.g., the composition is a gel. In an embodiment, the composition is a cream. In an embodiment, the biopolymer is chitosan. In an embodiment, the amount of biopolymer in the composition is equal to or less than about 5.0% w/w. In an embodiment, the amount of biopolymer in the composition is in a range recited herein for this ingredient. In an embodiment, the hydrophilic solvent is glycerin and propylene glycol. In an embodiment, the amount of each hydrophilic solvent is in a range recited herein for these solvents. In an embodiment, the hydrogenated vegetable oil is polyoxyl 40 hydrogenated castor oil. In an embodiment, the polyoxyl 40 hydrogenated castor oil is present in the composition in an amount of between about 0.5-5.0% w/w. In an embodiment, the amount of polyoxyl 40 hydrogenated castor oil in the composition is in a range recited herein for this ingredient.

In an embodiment, the composition comprises fusidic acid, greater than 1% w/w or at least about 1.1% w/w of a biopolymer, one or more hydrophilic solvents selected from glycerin, propylene glycol and both glycerin and propylene glycol, a hydrogenated vegetable oil, an oil mixture, collagen, and/or an emulsifier comprised of a fatty alcohol and an ethoxylated fatty alcohol. In an embodiment, the composition is translucent and/or semi-solid; e.g., the composition is a gel. In an embodiment, the composition is a cream. In an embodiment, the biopolymer is chitosan. In an embodiment, the amount of biopolymer in the composition is equal to or less than about 5.0% w/w. In an embodiment, the amount of biopolymer in the composition is in a range recited herein for this ingredient. In an embodiment, the hydrophilic solvent is glycerin and propylene glycol. In an embodiment, the amount of each hydrophilic solvent is in a range recited herein for these solvents. In an embodiment, the hydrogenated vegetable oil is polyoxyl 40 hydrogenated castor oil. In an embodiment, the polyoxyl 40 hydrogenated castor oil is present in the composition in an amount of between about 0.5-5.0% w/w. In an embodiment, the amount of polyoxyl 40 hydrogenated castor oil in the composition is in a range recited herein for this ingredient. In an embodiment, the oil mixture in the composition is comprised of a mineral oil and white petrolatum. In an embodiment, the oil mixture is present in the composition in an amount between about 8-15% w/w. In an embodiment, the mineral oil to white petrolatum in the oil mixture is in a ratio of between 1:1 to 0.5:1. In an embodiment, the ratio of mineral oil to white petrolatum in the oil mixture is between about 1:1 to 0.5:1, 0.9:1 to 0.5:1, 0.8:1 to 0.5:1, 0.7:1 to 0.5:1 or 0.65:1 to 0.55:1. In an embodiment, the composition comprises between about 2.0-8.0% w/w mineral oil. In another embodiment, the amount of mineral oil in the composition is in a range recited herein for this ingredient.

In an embodiment, the composition comprises fusidic acid, greater than 1% w/w or at least about 1.1% w/w of a biopolymer, one or more hydrophilic solvents selected from glycerin, propylene glycol and both glycerin and propylene glycol, a hydrogenated vegetable oil, an oil mixture, collagen, and/or an emulsifier comprised of a fatty alcohol and an ethoxylated fatty alcohol. In an embodiment, the composition is translucent and/or semi-solid; e.g., the composition is a gel. In an embodiment, the composition is a cream. In an embodiment, the biopolymer is chitosan. In an embodiment, the amount of biopolymer in the composition is equal to or less than about 5.0% w/w. In an embodiment, the amount of biopolymer in the composition is in a range recited herein for this ingredient. In an embodiment, the hydrophilic solvent is glycerin and propylene glycol. In an embodiment, the amount of each hydrophilic solvent is in a range recited herein for these solvents. In an embodiment, the hydrogenated vegetable oil is polyoxyl 40 hydrogenated castor oil. In an embodiment, the polyoxyl 40 hydrogenated castor oil is present in the composition in an amount of between about 0.5-5.0% w/w. In an embodiment, the amount of polyoxyl 40 hydrogenated castor oil in the composition is in a range recited herein for this ingredient. In an embodiment, the oil mixture in the composition is comprised of a mineral oil and white petrolatum. In an embodiment, the oil mixture is present in the composition in an amount between about 8-15% w/w. In an embodiment, the mineral oil to white petrolatum in the oil mixture is in a ratio of between 1:1 to 0.5:1. In an embodiment, the ratio of mineral oil to white petrolatum in the oil mixture is between about 1:1 to 0.5:1, 0.9:1 to 0.5:1, 0.8:1 to 0.5:1, 0.7:1 to 0.5:1 or 0.65:1 to 0.55:1. In an embodiment, the composition comprises between about 2.0-8.0% w/w mineral oil. In another embodiment, the amount of mineral oil in the composition is in a range recited herein for this ingredient. In an embodiment, collagen in present in the composition as marine collagen. In an embodiment, collagen is present in the composition as hydrolyzed marine collagen. In an embodiment, hydrolyzed marine collagen is present in the composition in an amount of between about 0.05-0.8% w/w. In an embodiment, the amount of hydrolyzed marine collagen in the composition is in a range recited herein for this ingredient.

In an embodiment, the composition comprises fusidic acid, greater than 1% w/w or at least about 1.1% w/w of a biopolymer, one or more hydrophilic solvents selected from glycerin, propylene glycol and both glycerin and propylene glycol, a hydrogenated vegetable oil, an oil mixture, collagen, and/or an emulsifier comprised of a fatty alcohol and an ethoxylated fatty alcohol. In an embodiment, the composition is translucent and/or semi-solid; e.g., the composition is a gel. In an embodiment, the composition is a cream. In an embodiment, the biopolymer is chitosan. In an embodiment, the amount of biopolymer in the composition is equal to or less than about 5.0% w/w. In an embodiment, the amount of biopolymer in the composition is in a range recited herein for this ingredient. In an embodiment, the hydrophilic solvent is glycerin and propylene glycol. In an embodiment, the amount of each hydrophilic solvent is in a range recited herein for these solvents. In an embodiment, the hydrogenated vegetable oil is polyoxyl 40 hydrogenated castor oil. In an embodiment, the polyoxyl 40 hydrogenated castor oil is present in the composition in an amount of between about 0.5-5.0% w/w. In an embodiment, the amount of polyoxyl 40 hydrogenated castor oil in the composition is in a range recited herein for this ingredient. In an embodiment, the oil mixture in the composition is comprised of a mineral oil and white petrolatum. In an embodiment, the oil mixture is present in the composition in an amount between about 8-15% w/w. In an embodiment, the mineral oil to white petrolatum in the oil mixture is in a ratio of between 1:1 to 0.5:1. In an embodiment, the ratio of mineral oil to white petrolatum in the oil mixture is between about 1:1 to 0.5:1, 0.9:1 to 0.5:1, 0.8:1 to 0.5:1, 0.7:1 to 0.5:1 or 0.65:1 to 0.55:1. In an embodiment, the composition comprises between about 2.0-8.0% w/w mineral oil. In another embodiment, the amount of mineral oil in the composition is in a range recited herein for this ingredient. In an embodiment, collagen in present in the composition as marine collagen. In an embodiment, collagen is present in the composition as hydrolyzed marine collagen. In an embodiment, hydrolyzed marine collagen is present in the composition in an amount of between about 0.05-0.8% w/w. In an embodiment, the amount of hydrolyzed marine collagen in the composition is in a range recited herein for this ingredient. In an embodiment, the emulsifier in the composition is the fatty alcohol cetostearyl alcohol, polyoxyl 20 cetostearyl ether, or a mixture of cetostearyl alcohol and polyoxyl 20 cetostearyl ether. In an embodiment, the composition comprises between about 5-9% w/w cetostearyl alcohol. In an embodiment, the amount of cetostearyl alcohol in the composition is in a range recited herein for this ingredient. In an embodiment, the amount of polyoxyl 20 cetostearyl ether in the composition is between about about 0.5-5.0% w/w. In an embodiment, the amount of polyoxyl 20 cetostearyl ether in the composition is in a range recited herein for this ingredient.

In an embodiment, a composition comprising 2% w/w fusidic acid, 1.25% w/w chitosan, 7% w/w glycerin, 16% w/w propylene glycol, 2% w/w polyoxyl 40 hydrogenated castor oil, 5% w/w light mineral oil, 7.5% w/w white petrolatum, 0.2% w/w marine hydrolyzed collagen, 2% w/w polyoxyl 20 cetostearyl ether, and/or 7.5% cetostearyl alcohol is provided. In an embodiment, the composition further comprises one or more of water, lactic acid, benzoic acid, butylated hydroxytoluene, edetate disodium, and/or dibasic sodium phosphate. In an embodiment, fusidic acid is formed in the composition in situ by reaction of sodium fusidate and nitric acid. In an embodiment, lactic acid is present in an amount of between 1-1.5% w/w and reacts with chitosan in situ to form chitosan lactate. In an embodiment, the composition is translucent and/or semi-solid; e.g., the composition is a gel. In an embodiment, the composition is a cream.

### Studies in Support of the Methods of Treatment

Examples 1 and 11 describe manufacture of exemplary compositions comprised of fusidic acid (2 wt %) and chitosan. The composition of Example 1 was used in studies to evaluate its effectiveness in treating skin disorders described herein. The composition of Example 11 is translucent and/or semi-solid and was used in studies to treat skin disorders, such as in the study described in Example 12.

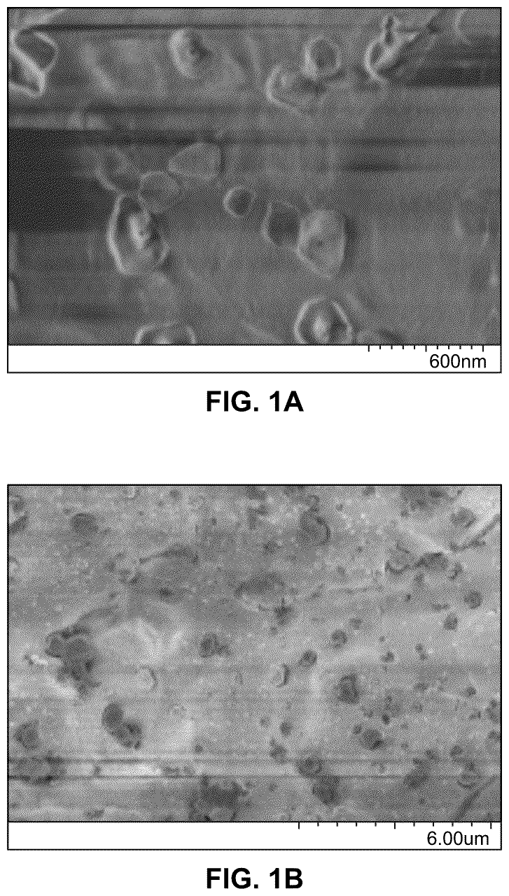

Example 2 describes microscopic inspection of a topical composition described herein. The antibacterial drug in the composition, fusidic acid, is present as a crystalline component. Efficacy depends in part on its surface-volume ratio, where a smaller the crystallite size with its larger surface area can provide better efficacy. As described in Example 1, the topical composition is prepared with sodium fusidate crystals having a mean diameter of several microns. Transformation from sodium fusidate to fusidic acid in situ results in a decrease in crystal size, with a concomitant surface to volume ratio increase. An exemplary cryo-SEM image of a shock-frozen sample of the composition is shown in FIG. 1A. The mean diameter of the crystals in this sample was determined by measuring the total area of the crystal assuming circular geometry, to provide an average diameter for a circular object of approximately 200 nm. In another sample of the composition, the crystal size was determined to be in the range of about 200-400 nm, as seen in FIG. 1B. The range of mean diameter of the fusidic acid crystals in the composition is attributed to a local variation of crystal size depending on the area where the measurement took place on the freeze fractured surface.

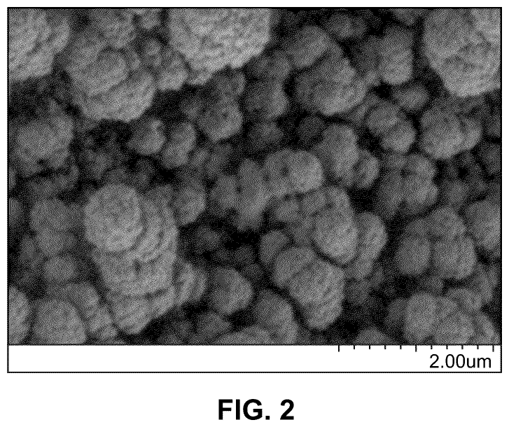

Another sample of the topical composition of Example 1 was applied to a freezing device and plunged into slushed nitrogen, then fractured in vacuum, followed treating to sublime water in order to reveal morphological structures in the composition. FIG. 2 is an image of a sample treated by sublimation to remove most water from the composition. The image shows the oil phase morphology of the composition that remains and the phase structure of the oil-in-water emulsion.

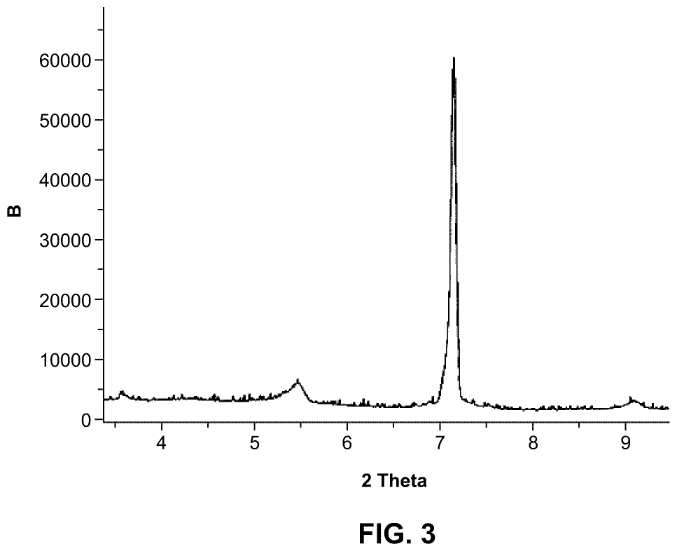

The topical composition of Example 1 was also inspected by x-ray diffraction. An x-ray diffractogram of the composition is shown in FIG. 3, and a distinct peak at around 7° that corresponds to the fusidic acid crystals is seen. As described in Example 2, the average crystallite size was determined from the full-width-at-half-maximum of the peak at 7.14° to be about 120 nm. This measurement yields the size of the crystallites and not the size of the crystals, because the x-ray measurement reflects only the perfect crystal size and ignores total morphology.

From the SEM and x-ray measurements it was concluded that the fusidic acid crystals in the topical composition have a diameter, in an embodiment, of between about 100-800 nm, 150-600 nm, 200-600 nm, 200-500 nm, or 200-400 nm. In an embodiment, the diameter is an average diameter, and in another embodiment, the diameter is a mean diameter.

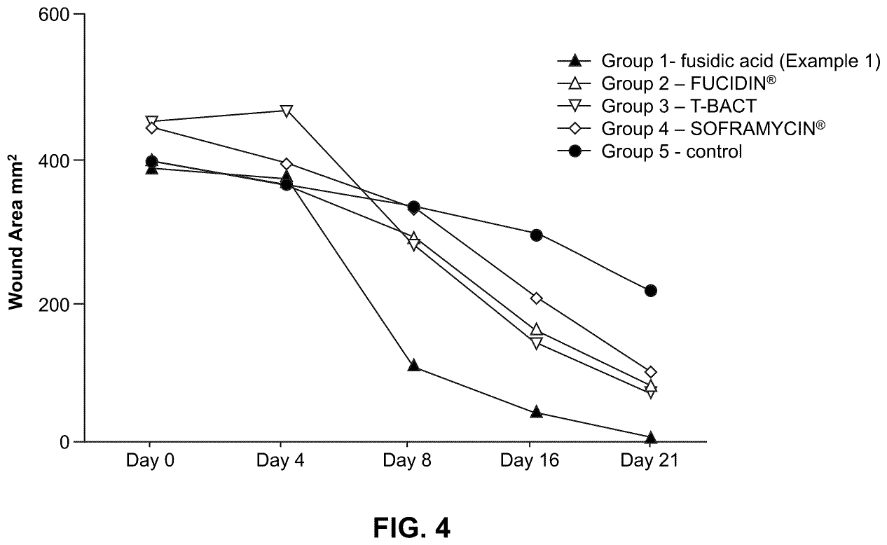

A study was also conducted to evaluate the composition of Example 1 with fusidic acid and chitosan in treating burn wounds. As described in Example 5, the fusidic acid composition of Example 1 was comparted to commercially available reference products Fucidin™ (Leo-Ranbaxy, India), T-Bact™ ointment (GSK), and SOFRAMYCIN® (Sanofi-Aventis). The formulations were applied to the wounds once daily as a single layer for 21 days. Wound areas were assessed by measuring the area of the wound from tracings at 4, 8, 16 and 21 days. (Bairy et al., India J Exp Biol 35:70-72 (1997)). The results are shown in FIG. 4.

As seen in FIG. 4, wound contraction results revealed that treatment with the fusidic acid composition of Example 1 (closed triangles) produced maximum wound contraction between days 4 to 8 (70.53%). Similar findings are recorded in the other groups throughout the experimental period and by day 21, 100% wound contraction with scar formation was observed in the group of rats treated with the fusidic acid composition of Example 1 (closed triangles) when compared to untreated animals (control, circles) and other treated groups (Fucidin cream, open triangles; T-Bact™, inverted triangles; and SOFRAMYCIN, diamonds).

Studies were also performed to determine activity of the fusidic acid composition of Example 1 (2% fusidic acid) against Methicillin resistant (MRSA) Staphylococcus aureus. As described in Example 6, the minimum inhibition concentrations (MIC 50/MIC 90) of mupirocin and fusidic acid against Methicillin resistant Staphylococcus aureus were determined. The results in Table 6-1 to 6-3 show the potency of fusidic acid in the composition of Example 1 compared to antimicrobials in other topical products. Example 7 details a further study to demonstrate activity of the fusidic acid composition of Example 1 (2% fusidic acid) against Methicillin resistant (MRSA) Staphylococcus aureus. The data in Example 7 shows that a fusidic acid cream described herein was comparatively better than comparative treatments, such as Fucidin™ cream and T-Bact™ phenotypically and genotypically and was less likely to support the development of resistant mutants when used topically.

Accordingly, in an embodiment, a method for reducing likelihood of skin infection is provided. The method comprises topically applying the composition to skin of a person in need, where the subject in an embodiment is a person suffering from EB. In an embodiment, topically applying achieves a reduction in likelihood of creating or developing antibiotic resistant mutants, such as a reduction in Staphylococcus aureus mutants.

Example 8 describes a study to evaluate and compare the fusidic acid composition with a biopolymer (chitosan), i.e., 2% fusidic acid cream of Example 1, in treating wounds and in resolving itch and pain associated with the wound. The data in Example 8 demonstrates that the composition described herein was more effective than the comparator compositions in achieving clinical improvement or resolution of skin infections. The fusidic acid composition with a chitosan polymer provided superior patient centric therapeutic benefits over the other products tested.

Example 9 describes a study to evaluate the efficacy the fusidic acid composition with a biopolymer (chitosan), i.e., 2% fusidic acid cream of Example 1, in wound healing. For this study a porcine full thickness wound model was used. The composition of Example 1 was compared to a composition with chitosan only, a composition with sodium fusidate only, and a placebo composition. The data is shown in Tables 9-4, 9-5, 9-6 and 9-7 of Example 9. The data from this 28 day study showed that the composition with fusidic acid and a biopolymer healed full thickness wounds more rapidly than a control, placebo composition lacking the fusidic acid and biopolymer. A composition with only chitosan as the active ingredient also provided more rapid wound healing than the control, placebo composition. The composition with fusidic acid and a biopolymer and the composition with only chitosan provided better histopathological, epithelialisation and wound healing scores than the compositions with only fusidic acid (Composition 3).

Example 10 describes a study using a porcine partial thickness wound model to evaluate the wound healing of the compositions described in Example 9. The study evaluated wound area reduction, histological confirmation of the wound healing, antibacterial effect through reduction of colony forming units and quality of wound scarring for the three test compositions and for the placebo composition (Table 9-1). As detailed in Example 10, in the porcine wound healing model study twelve partial thickness wounds were created on the back of each of eight pigs seven days prior to application of the compositions with inoculation of bacterial culture to create infection. There were four arms dedicated to each test composition comprising three wounds per composition per animal. The compositions were applied on the wounds starting on test day 0 and again on test days 7, 14, and 21. On test day zero all the wounds showed presence of biofilm as a confirmation of infection.

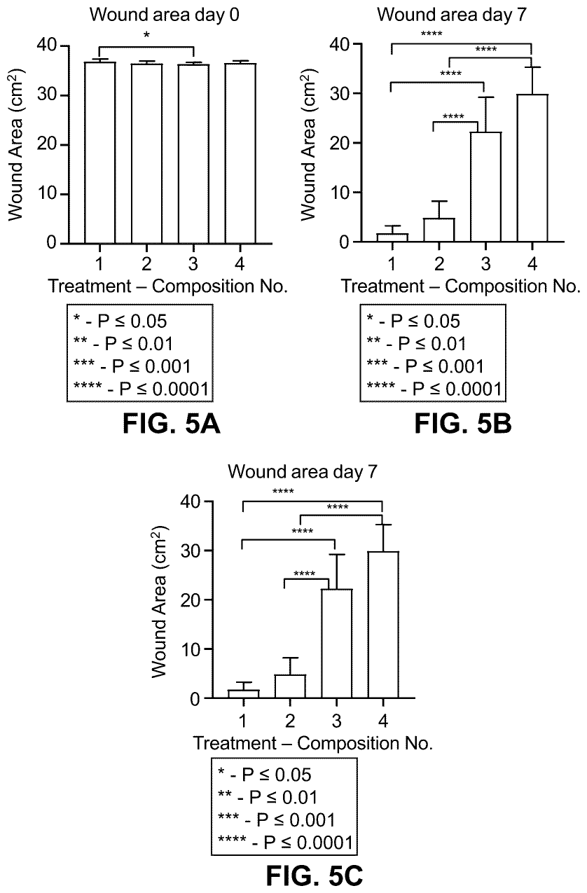

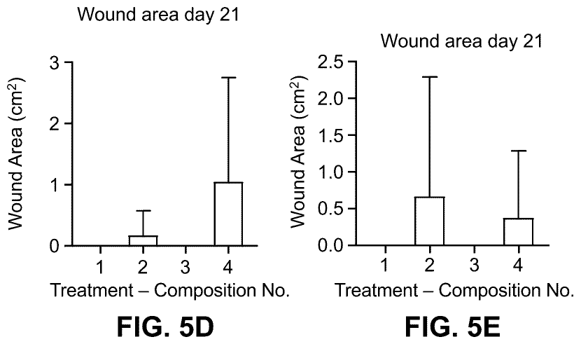

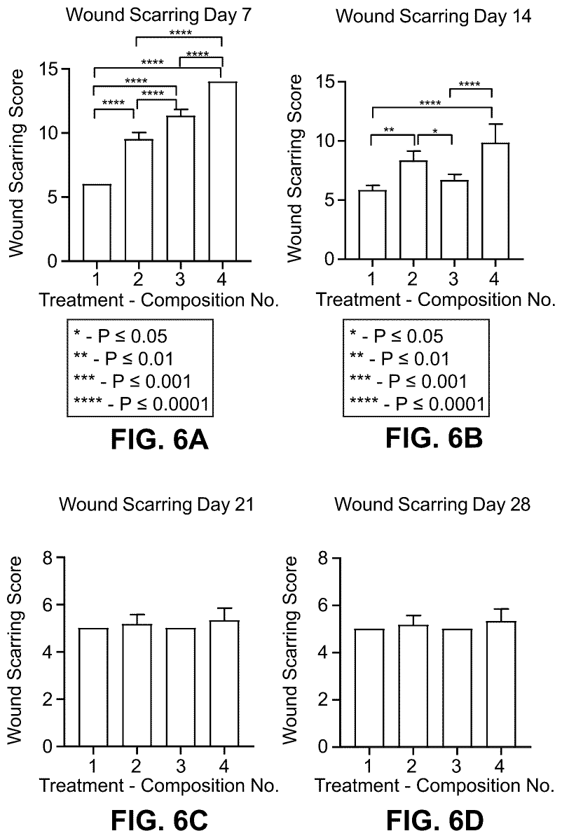

Planimetry measurements were taken together with CFU counts to evaluate the wound healing 7, 14, 21 and 28 days after initial application of each composition. Results are shown in FIGS. 5A-5D and FIG. 6. On day 7 following the removal of the sterile dressing from left side and right side wounds of each animal showed that all the wounds were wet with presence of pus exudates with initiation of scab formation. Planimetry measurements for wound size (wound size reduction) revealed that largest reduction or contraction of wound size in wounds treated with the test composition comprising 2% fusidic acid with chitosan biopolymer, where the treated wounds had 1.69±0.1 cm² mean area. Wounds treated with chitosan cream (composition 2) had a 4.77±2.33 cm² mean area at test day 7, and wounds treated with the fusidic acid cream (composition 3) had a 22.22±8.15 cm² mean area. Wounds treated with the placebo cream shows the least contraction was or reduction of wound size with 29.85±16.50 cm² mean area of wounds.

Two animals (P3 and P4) were dedicated for planimetry measurements on day 14. Results are seen in FIG. 5C. Planimetry measurements for wound size (wound size reduction) revealed the greatest reduction or contraction of the wound in wounds treated with the composition comprising 2% fusidic acid with chitosan biopolymer (composition 1), with 0.62±0.40 cm² mean area of wounds, followed by wounds treated with the fusidic acid cream (composition 3) with 4.86±1.90 cm², followed by wounds treated with chitosan cream (composition 2) with 9.59±2.33 cm² mean area of wounds. The least contraction was or reduction of wound size was recorded in the wounds treated with placebo (composition 4), with 27.74±14.83 cm² mean area of wounds.

Two animals (animals P5 and P6) were dedicated for planimetry measurements on day 21 and two animals (animals P7 and P8) were dedicated for planimetry measurements on day 28. Results are seen in FIGS. 5D-5E. Wounds treated with the composition comprising 2% fusidic acid with chitosan biopolymer (composition 1) and with the fusidic acid cream (composition 3) had healed. Wounds treated with chitosan cream (composition 2) were more healed than wounds treated with placebo (composition 4).

An evaluation of wound scarring was done using a visual analogue score, set forth in Table 9-2 of Example 9 that evaluates color, surface, contour, distortion and texture of the wounds. Results are shown in FIGS. 6A-6D. As per the scoring criteria for this test, a lower visual analogue score indicates better healing/less scarring. Wounds treated with the composition comprising 2% fusidic acid with chitosan biopolymer (composition 1) and with the fusidic acid cream (composition 3) clearly showed better healing/less scarring than wounds treated with chitosan cream (composition 2) or with placebo, until study day 14. On study day 21 and day 28 almost a similar sum of the visual analogue score for all wounds was observed.

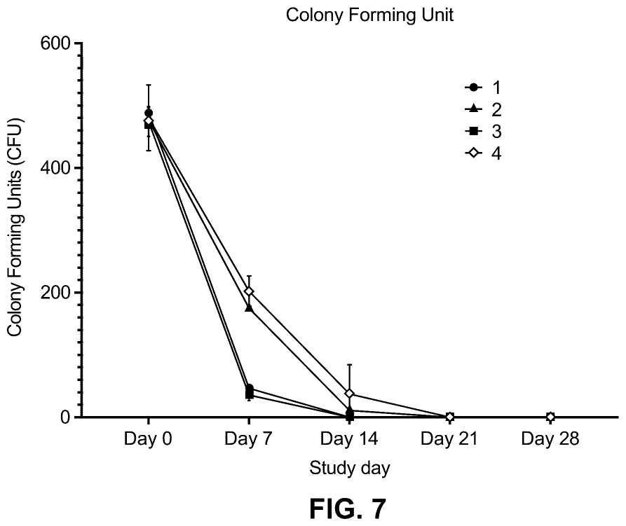

With regard to biofilm presence in the wounds, results are shown in FIG. 7. FIG. 7 is a graph of colony forming units (CFU) as a function of study day from swabs taken from wounds treated with the fusidic acid and chitosan composition of Example 1 (composition 1, closed circles), a cream with chitosan (composition 2, closed triangles), a cream with fusidic acid (composition 3, closed squares) and placebo (composition 4, open diamonds). On day zero all the wounds showed mean OD value 0.299, where greater than 0.120 that indicates presence of biofilm. One sample per wound was collected on day 0 and on necropsy days (day 7, day 14, day 21 and day 28, serially diluted from 10¹ to 10⁶ dilutions, plated and incubated for CFU measurements. The 10⁶ dilution showed separate countable colonies and clear clones were considered for colony counting results evaluation. Wounds treated with the composition comprising 2% fusidic acid with chitosan biopolymer (composition 1) and with the fusidic acid cream (composition 3) showed least colony forming units on day 7 and day 14 as compared to wounds treated with chitosan cream (composition 2) or with placebo. No colony forming units on day 21 and day 24 in any of the treated wounds were observed.

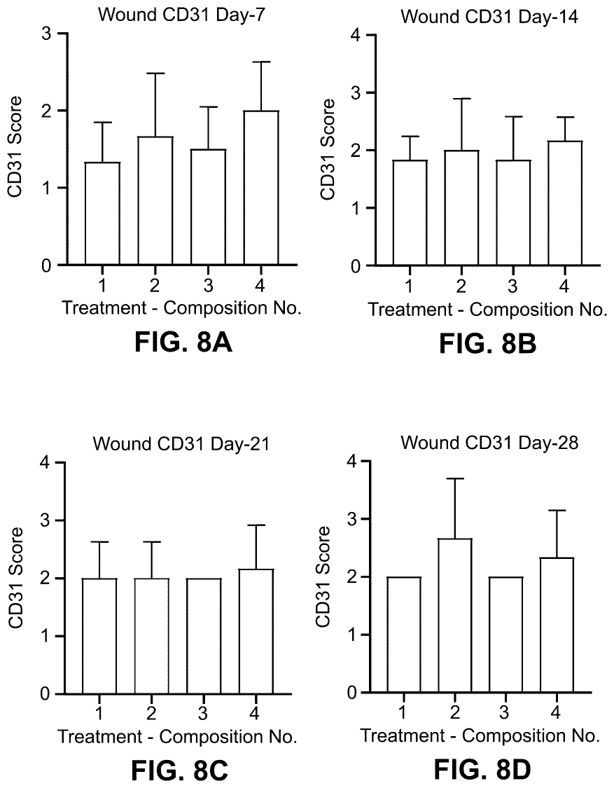

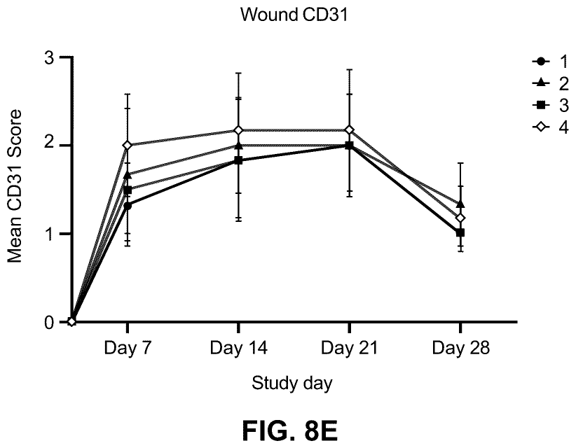

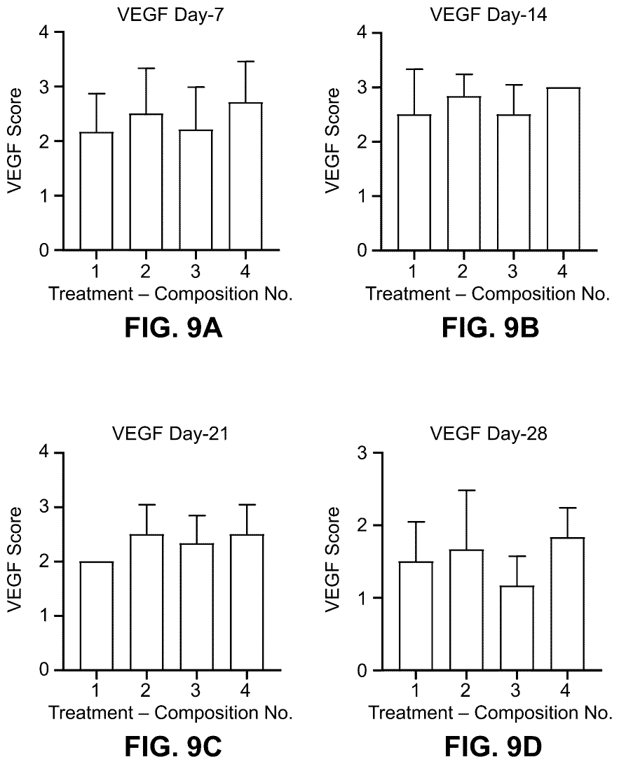

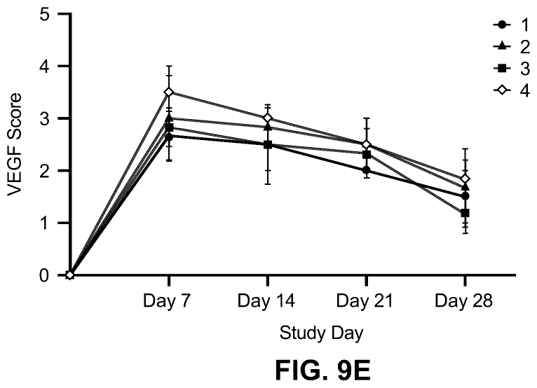

Wound sections were subjected to immunohistochemistry staining to detect CD31 and VEGF markers and scored for positively stained area using the scoring system in Table. 10-3. Results for CD31 are shown in FIGS. 8A-8E and results for VEGF are shown in FIGS. 9A-9E. As seen in FIGS. 8A-8E, the mean CD31 score of wounds treated with compositions other than placebo increased from day 7 (FIG. 8A) to 21 (FIG. 8C) and then reduced on day 28 (FIG. 8D). This increase in CD31 indicates neovascularization in the initial stage of wound healing followed by the remodeling (restoration) phase of wound healing. In these figures, the fusidic acid and chitosan composition of Example 1 is composition 1 (closed circles in FIG. 8E), the cream with chitosan is composition 2 (closed triangles in FIG. 8E), the cream with fusidic acid is composition 3 (closed squares in FIG. 8E)) and placebo is composition 4 (open diamonds in FIG. 8E).

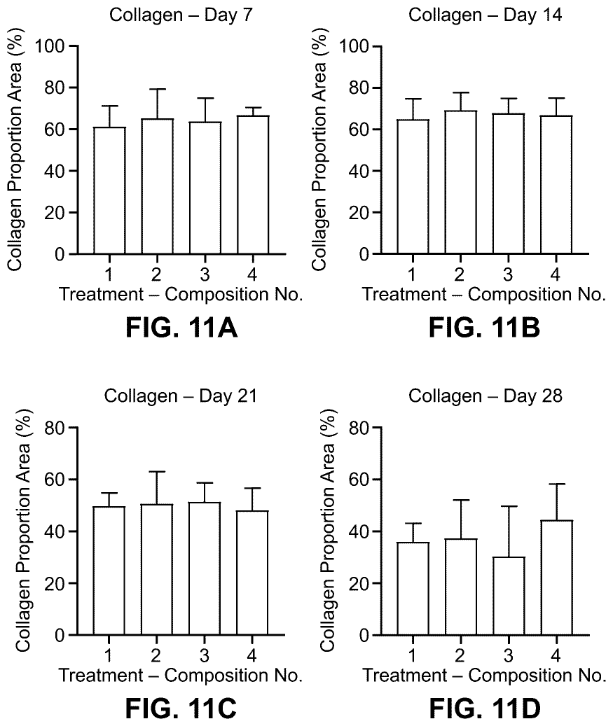

As seen in FIGS. 9A-9E, the mean score of VEGF was decreased from day 7 (FIG. 9A) to 28 (FIG. 9D) in wounds treated with compositions other than placebo. Wounds treated with the composition comprising 2% fusidic acid with chitosan biopolymer (composition 1) and with the fusidic acid cream (composition 3) showed a faster reduction in VEGF level. The reduction in VEGF level was faster in the cream with chitosan (composition 2) than in placebo, composition 4. These results reflect reorganization or reduction of dermal collagen and the results correlate with collagen proportion area, discussed below with respect to FIGS. 11A-11D.

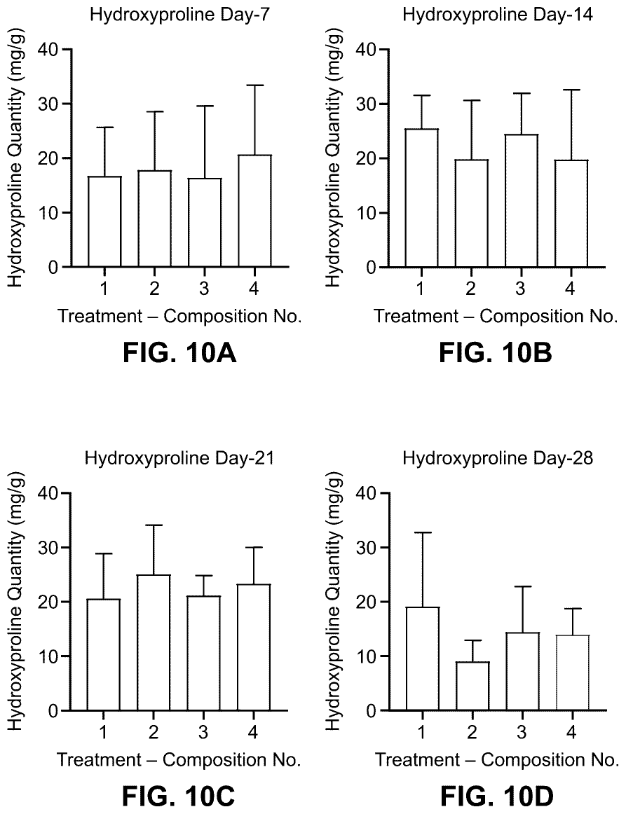

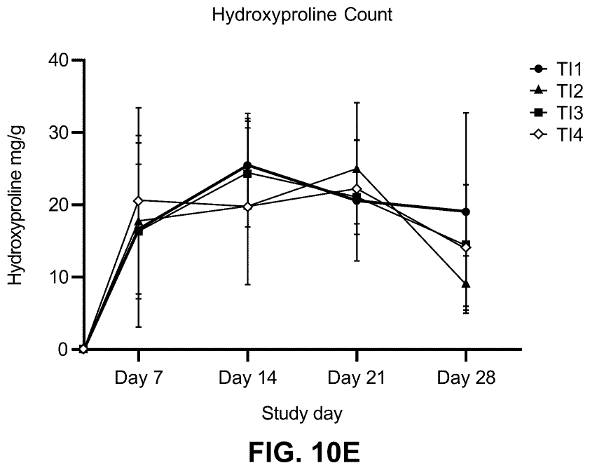

Results for immunohistochemical analysis for hydroxyproline are shown in FIGS. 10A-10D, which show bar graphs of hydroxyproline amount (mg/g) for wounds in the porcine partial thickness model treated with the fusidic acid and chitosan composition of Example 1 (Composition 1), a cream with chitosan (composition 2), a cream with fusidic acid (composition 3) and placebo (composition 4), at study day 7 (FIG. 10A), study day 14 (FIG. 10B), study day 21 (FIG. 10C), and study day 28 (FIG. 10D). The mean hydroxyproline content was increased from day 7 to 21 and then reduced on day 28 in all the test item treated wound sites.

FIG. 10E is a graph of the hydroxyproline amount (mg/g) as a function of study day for wounds treated with the fusidic acid and chitosan composition of Example 1 (composition 1, closed circles), a cream with chitosan (composition 2, closed triangles), a cream with fusidic acid (composition 3, closed squares) and placebo (composition 4, open diamonds).

Wound sections stained with Sirius Red were analyzed for percent collagen proportion area (% CPA). FIGS. 11A-11D are bar graphs of collagen count for wounds in a porcine partial thickness model treated with the compositions and with placebo. It was observed that restoration of rete ridges, orientation, density and maturity of collagen fiber was better in wounds treated with the composition comprising 2% fusidic acid with chitosan biopolymer (composition 1) and with the fusidic acid cream (composition 3), followed by wounds treated with the chitosan cream (composition 2), from day 7 to day 28. These results indicate earlier and faster healing of the wounds treated with the composition comprising 2% fusidic acid with chitosan biopolymer (composition 1) and with the fusidic acid cream (composition 3).

The excess collagen reduction was better in wounds treated with the composition comprising 2% fusidic acid with chitosan biopolymer (composition 1) and with the fusidic acid cream (composition 3). Wounds treated with the cream with chitosan (composition 2) showed an excess collagen reduction better than wounds treated with placebo (composition 4). These results indicate early and faster maturation or reorganization of dermal collagen of wound sites treated with the composition comprising 2% fusidic acid with chitosan biopolymer (composition 1) and with the fusidic acid cream (composition 3) compared to wounds treated with placebo.

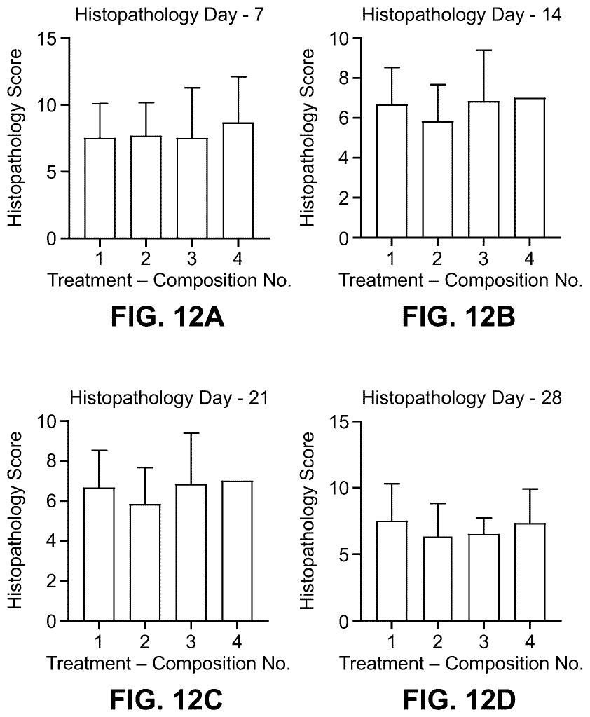

Wound sections stained with H&E were scored for of restoration of rete ridges, orientation, density and maturity of collagen fiber, using the scoring system set forth in Table 9-3. Results are shown in FIGS. 12A-12D. It was observed that restoration of rete ridges, orientation, density and maturity of collagen fiber was better wounds treated with the composition comprising 2% fusidic acid with chitosan biopolymer (composition 1) and with the fusidic acid cream (composition 3), followed wounds treated with chitosan cream (composition 2), from day 7 (FIG. 12A) to day 28 (FIG. 12D). These results indicate earlier and faster healing of the wounds treated with the composition comprising 2% fusidic acid with chitosan biopolymer (composition 1) and with the fusidic acid cream (composition 3), than wounds treated with chitosan cream or with placebo. In dermis, wound healing was indicated by horizontal orientation, fascicle pattern of fibers, maturation of collagen and reduction in stromal components, as the days progressed from 7 to 28. Neoepithelialization is characterized by gradual increase in the covering of dermis by newly formed epithelium from day 7 to 28 and was observed in all treated wounds.

Example 12 describes a study to assess wound healing in a diabetic porcine full thickness wound model over a 43 day period. The data from the study demonstrated that the compositions with fusidic acid and chitosan (Composition 12-1), chitosan (Composition 12-2), fusidic acid (Composition 12-3) provide, relative to placebo (Composition 12-4), improved wound healing, as measured by several parameters such as wound area reduction. These compositions also demonstrated superior wound healing relative to placebo when evaluated by histopathological analysis. The compositions also demonstrated improved, relative to placebo, effect on collagen fiber parameters, neovascularization, inflammation and scab formation, each indicative of the superior wound healing achieved by the compositions.

Examples 13 and 14 each describe a study in subjects with EB treated with a composition of Example 11 that comprises fusidic acid and chitosan.

### IV. Examples

The following examples are illustrative in nature and are in no way intended to be limiting.

### Example 1

### Preparation of Exemplary Composition

A topical composition comprising fusidic acid was prepared as described in U.S. Pat. No. 8,895,542 (which is incorporated by reference herein) with the components and in the amount indicated in Table 1-1. Fusidic Acid is formed in situ in the composition via reaction of sodium fusidate and nitric acid. Chitosan lactate is formed in situ in the composition via reaction of chitosan and lactic acid.

TABLE 1-1
Composition with Fusidic Acid

| | Amount | Category/ |
|---|---|---|
| Ingredients | % w/w | Role of Ingredient |
| Sodium fusidate equivalent to | 2.08 | Anti-bacterial |
| Fusidic Acid | | |
| Chitosan | 0.25 | Wound Healing Agent |
| Edetate disodium | 0.10 | Chelating agent |
| Lactic acid¹ | 0.14 | Pharmaceutical aid |
| White petrolatum | 8.5 | Pharmaceutical aid |
| Cetostearyl alcohol | 5.1 | Emulsifying agent |
| Polyoxyl 20 cetostearyl ether | 1.8 | Emulsifying agent |
| Sorbitan monostearate | 0.70 | Non-ionic surfactant |
| Stearyl alcohol | 3.2 | Emulsifying agent |
| Butylated hydroxytoluene | 0.01 | Antioxidant |
| Propylene glycol | 25.0 | Co-solvent |
| Nitric acid² | 0.284 | Pharmaceutical aid |
| Dibasic sodium phosphate | 0.05 | Buffering agent |
| Benzoic acid | 0.20 | Anti-microbial preservative |
| Purified water | q.s. | Solvent |

¹Added as a processing aid for the formation of chitosan lactate from chitosan.
²Added as a processing aid for the formation of fusidic acid from sodium fusidate.

### Example 2

### Microscopic Analysis of an Exemplary Composition

A topical composition prepared as described in Example 1 was observed by cryogenic scanning electron microscopy (cryo-SEM) to determine the crystal size of the active component fusidic acid. The composition was also analyzed by x-ray diffraction.

Cryo-SEM was performed with a Hitachi SU 8000 SEM equipped with a Quorum KX 1250 cryo preparation and transfer device. A small amount of the composition was applied to a freezing device and plunged into slushed nitrogen. Afterwards the sample was fractured in vacuum, followed by a short procedure to sublime water in order to reveal morphological structures in the composition. For some preparations, iridium was sputtered onto the frozen surface of the sample to increase surface conductivity of the specimen.

From several SEM images crystalline objects that match the requirements as to be identified as crystals of fusidic acid were identified. An exemplary cryo-SEM image of a shock-frozen sample of the composition is shown in FIG. 1A. The mean diameter of the crystals in this sample was determined by measuring the total area of the crystal assuming circular geometry, to provide an average diameter for a circular object. The mean diameter of the crystals as identified by SEM was approximately 200 nm. In another sample of the composition, the crystal size was determined to be in the range of below about 400 nm, as seen in FIG. 1B. The range of mean diameter of the fusidic acid crystals in the composition is attributed to a local variation of crystal size depending on the area where the measurement took place on the freeze fractured surface.

Another sample of the topical composition of Example 1 was applied to a freezing device and plunged into slushed nitrogen, then fractured in vacuum, followed treating to sublime water in order to reveal morphological structures in the composition. FIG. 2 is an image of a sample treated by sublimation to remove most water from the composition. The image shows the oil phase morphology of the composition that remains and the phase structure of the oil-in-water emulsion.

The composition of Example 1 was also inspected by x-ray diffraction. An x-ray diffractogram of the composition is shown in FIG. 3, and a distinct peak at around 7° that corresponds to the fusidic acid crystals is seen. Based on the Scherrer-formula, an average crystallite size can be determined from the full-width-at-half-maximum (FWHM) of one crystal peak from the x-ray diffractogram. The FWHM was determined to be 0.0013 for the peak at 7.14°. From this the mean crystallite size was calculated from the Scherrer formula to be L=120 nm. This measurement yields the size of the crystallites and not the size of the crystals, because the x-ray measurement reflects only the perfect crystal size and ignores total morphology. Thus, the fusidic acid crystals in the composition can be larger than 120 nm, but there are no more single crystals which gives the lower value of mean crystallite size.

### Example 3

### Method of Treating Wounds

A topical composition of Example 1 containing fusidic acid and chitosan lactate was prepared. For comparison with the Example 1 composition, three compositions were prepared or obtained: (1) a comparative composition with no chitosan was prepared; (2) fucidin cream was obtained commercially (Fucidin™, Leo-Ranbaxy, India); and (3) Fucidin® with chitosan biopolymer added.

Thirty male Wistar rats of 150-200 grams and 6 months old were obtained and housed individually under controlled conditions of temperature 23±2° C., humidity of 50±5% and 10-14 hours of light and dark cycle, respectively. The animals had free access to sterile food (animal chow) and water ad libitum.

Partial thickness burn wounds were inflicted on overnight starved animals under ketamine anesthesia (24 mg/kg/i.p), by pouring hot molten wax at 80° C. into a metal cylinder of 300 mm² circular opening placed on shaven back of the rat. Immediately after the injury and on subsequent days Ringer Lactate (1 ml/kg) was administered i.p., for resuscitation. Wound contraction was monitored by measuring wound area, planimetrically, on the alternate days until the wounds were completely healed. The percent of wound contraction on day 4, 8, 12 and 16 were calculated. Time taken for full epithelization was measured by recording the days required for fall of scab leaving no raw wound behind. Apart from the drugs under investigation no local/systemic chemotherapeutic cover was provided to animals.

Animals showing signs of infection were excluded from the study and replaced with fresh animal. The rats were randomly divided into five treatment groups: Group 1 did not receive any drug and served as control group. Groups 2, 3, 4, 5, received, respectively, the test composition of Example 1, comparative test composition of Example 1 with no chitosan, fucidin cream Fucidin™ (Leo-Ranbaxy, India), and fucidin cream with added chitosan biopolymer, each once daily for 21 days or until complete healing, whichever was earlier. Results are shown in Tables 3-1 and 3-2.

TABLE 3-1
Time for Epithelization

| | Period of | |
|---|---|---|
| | epithelization | p-value |
| Test Article | (days) | (*significant) |
| Control (no treatment) | 22.31 ± 1.76 | |
| Fucidin cream (Leo-Ranbaxy) | 16.50 ± 1.45 | 0.027* |
| Fucidin cream (Leo-Ranbaxy) | 19.16 ± 1.10 | 0.428 |
| with added biopolymer | | |
| Fusidic acid composition, | 15.00 ± 1.03 | 0.004* |
| with no biopolymer | | |
| Fusidic acid composition | 14.00 ± 0.85 | 0.001* |
| (Example 1) | | |

TABLE 3-2
Percent of Wound Contraction

| | Percent of Wound Contraction | | | |
|---|---|---|---|---|
| | (p-value, * if significant) | | | |
| Test Article | Day 4 | Day 8 | Day 12 | Day 16 |
| Control (no treatment) | 14.21 ± 3.54 | 8.13 ± 0.77 | 33.18 ± 0.86 | 59.06 ± 4.27 |
| Fucidin cream (Leo- | 18.63 ± 3.10 | 48.70 ± 4.48 | 54.96 ± 4.58 | 85.26 ± 2.57 |
| Ranbaxy) | (0.816) | (0.0001*) | (0.014*) | (0.002*) |
| Fucidin cream (Leo- | 9.53 ± 2.58 | 30.23 ± 3.43 | 37.66 ± 2.48 | 72.56 ± 4.39 |
| Ranbaxy) with | (0.783) | (0.005*) | (0.950*) | (0.198) |
| biopolymer | | | | |
| Fusidic acid composition | 20.70 ± 2.03 | 34.83 ± 3.16 | 41.36 ± 4.69 | 77.66 ± 5.19 |
| with no biopolymer | (0.522) | (0.001*) | (0.685) | (0.036*) |
| Fusidic acid composition | 26.80 ± 2.99 | 35.16 ± 6.15 | 50.50 ± 6.84 | 84.10 ± 4.41 |
| (Example 1) | (0.037*) | (0.001*) | (0.070) | (0.003*) |

The data in Table 3-1 shows that the period of epithelization in the untreated, control group was 22.33±1.76 days. The composition of Example 1 with fusidic acid and chitosan biopolymer provided a 37% improvement in time (days) it took for epithelization compared to the untreated, control animals. The composition of Example 1 with fusidic acid but with no chitosan biopolymer provided a 33% improvement in time (days) it took for epithelization compared to the untreated, control animals. And the test composition of Example 1 with biopolymer and without biopolymer performed better than the commercially available Fucidin cream. Interestingly, addition of the chitosan biopolymer to the commercially available Fucidin cream resulted in a decrease performance of the cream, decreasing the days for epithelization from 16.5 days to 19.2 days.

With regard to wound contraction, the composition with fusidic acid and chitosan biopolymer (Example 1 composition) and the commercially available Fucidin cream provided similar response to wound contraction.

### Example 4

### Method of Treating Wounds

A topical composition of Example 1 comprising fusidic acid and chitosan lactate was prepared. For comparison with the Example 1 composition, several compositions listed in Table 4-1 were obtained.

Thirty male Wistar rats of 150-200 grams and 6 months old were obtained and housed individually under controlled conditions of temperature 23±2° C., humidity of 50±5% and 10-14 hours of light and dark cycle, respectively. The animals had free access to sterile food (animal chow) and water ad libitum.

Partial thickness burn wounds were inflicted as described in Example 3. Wound contraction was monitored by measuring wound area, planimetrically, on the alternate days until the wounds were completely healed, and time taken for full epithelization was measured by recording the days required for fall of scab leaving no raw wound behind, as described in Example 3. The wound closure time (WC50) was calculated by the method of Litchfield and Wilcoxon (J. Pharmacol. Exp. Ther., 95:99-135 (1949)).

Group 1 did not receive any drug and served as control group. Groups 2, 3, 4, 5, 6, and 7 received, respectively, topically the test composition of Example 1, an ointment with neomycin (Framyceti), calcium mupirocin cream, Fucidin™ cream, T-Bact™ ointment, or SOFRAMYCIN® cream, each once daily for 21 days or until complete healing, whichever was earlier.

Results are shown in Tables 4-1 and 4-2.

TABLE 4-1
Time for Epithelization

| | Period of epithelization | p-value |
|---|---|---|
| Formulation | (days) | (*significant) |
| 1 - Control (no treatment) | 21.7500 ± 0.25 | — |
| 2 - Fusidic acid composition | 17.2500 ± 1.50 | 0.042* |
| (Example 1) | | |
| 3 - Framycetin cream | 19.7500 ± 1.33 | 0.801 |
| 4 - Calcium mupirocin cream | 21.5000 ± 0.73 | 1.000 |
| 5 - Fucidin ™ cream (Leo- | 19.2500 ± 0.99 | 0.589 |
| Ranbaxy) | | |
| 6 - T-Bact ™ ointment(GSK) | 19.2500 ± 1.19 | 0.589 |
| 7 - SOFRAMYCIN ® cream | 23.5000 ± 0.32 | 0.882 |
| (Sanofi-Aventis) | | |

TABLE 4-2
Percent of Wound Contraction

| | Percent of Wound Contraction | | | |
|---|---|---|---|---|
| | (p-value, * if significant) | | | |
| Formulation | Day 4 | Day 8 | Day 12 | Day 16 |
| 1 - Control (no treatment) | 0.028 ± 3.76 | 15.63 ± 4.24 | 23.4 ± 3.44 | 58.1 ± 8.4 |
| 2 - Fusidic acid composition | 34.03 ± 5.66* | 53.4 ± 3.9* | 71.6 ± 7.67* | 92.4 ± 7.5* |
| (Example 1) | | | | |
| 3 - Framycetin cream | 0.56 ± 6.26 | 27.67 ± 5.0 | 45.7 ± 2.35 | 53.09 ± 7.4 |
| 4 - Calcium mupirocin cream | 2.27 ± 6.21 | 18.27 ± 5.13 | 30.1 ± 6.49 | 42.2 ± 5.81 |
| 5 - Fucidin ™ cream (Leo- | 8.27 ± 5.60 | 24.8 ± 7.4 | 37 ± 6.75 | 57.1 ± 10.37 |
| Ranbaxy) | | | | |
| 6 - T-Bact ™ ointment (GSK) | 17.16 ± 9.16 | 34.4 ± 6.16 | 49.7 ± 6.19 | 75.07 ± 8.22 |
| 7 - SOFRAMYCIN ® cream | 17.87 ± 4.46 | 32.3 ± 4.0 | 42.2 ± 4.82 | 63.28 ± 4.05 |
| (Sanofi-Aventis) | | | | |

The data in Table 4-1 shows that the fusidic acid composition significantly reduced the time for epithelialization when compared to the control group and the other creams tested. This is further reflected by histopathologic studies performed (data not shown). With regard to wound contraction, the data in Table 4-2 shows that the fusidic acid composition significantly improved the wound contraction when compared to the control group and the other creams tested. This is further reflected by histopathologic studies performed (data not shown). Taken together, the results in Tables 4-1 and 4-2 are clinically significant for burn patients, since the fusidic acid composition not only controls infection but also favors healing of burn wounds.

### Example 5

### Method of Treating Burn Wounds

Fifty Wistar rats of either sex weighing of 150-200 grams were obtained and housed individually under controlled conditions of temperature 22±4° C., humidity of 60±2% and 12 hours of light and dark cycles. The animals had free access to an unlimited supply of rodent pellet diet and sterilized drinking water ad libitum.

The rats were anaesthetized with thiopental sodium (45 mg/kg body weight) and the hairs on the back were clipped with electrical clippers. Burn wounds were created by pouring hot molten wax at 80° C. into a metal cylinder (300 mm area of circular openings and capacity to hold 4.6 g of wax) placed on the back of the rat. Upon solidification of the wax (approx. 8 minutes), the metal cylinder with wax adhered to skin was removed, leaving distinctly marked circular wounds of 300 mm area. Each animal was then placed in a separate cage for full recovery of anesthesia before being returned to holding rooms.

The formulations listed in Table 5-1 were applied to the wounds of rats in groups of ten per formulation once daily as a single layer for 21 days. No local or systemic chemotherapeutic agents were administered.

TABLE 5-1
Group No. (n = 10) and Test Formulations

| Group No. | Group No. - Formulation |
|---|---|
| 1 | Fusidic acid composition |
| | (Example 1) |
| 2 | Fucidin ™ cream (Leo-Ranbaxy) |
| 3 | T-Bact ™ ointment (GSK) |
| 4 | SOFRAMYCIN ® cream |
| | (Sanofi-Aventis) |
| 5 | Control (no treatment) |

Wound areas were assessed by measuring the area of the wound from tracings at 4, 8, 16 and 21 days. (Bairy et al., India J Exp Biol 35:70-72 (1997)). The results are shown in FIG. 4.

In addition, hydroxyproline content, a marker for wound healing, was assayed after 21 days of treatment. Hydroxyproline content was estimated by spectrophotometer method according to Reddy and Enwemeka, (Clin. Biochem. 29 (3): 225-229 (1996)). The skin samples were hydrolyzed by autoclaving at 1200° C. for 20 min, 450 μL of chloramine-T was added to the 50 μL of hydroxylate, mixed gently and the oxidation was allowed to proceed for 25 minutes at room temperature. 500 μL of Ehrlich's reagent (p-dimethyl aminobenzaldehyde in n-propanol/perchloric acid) was then added to each sample, mixed gently, and the chromophore was developed by incubating the mixture at 650° C. for 20 minutes. The absorbance of a reddish-purple complex was measured spectrophotometrically at 550 nm. A standard curve was prepared with hydroxyproline (1 mg/mL). Hydroxyproline content in the skin samples were expressed in μg/gm. Results are shown in Table 1.

### Example 6

### Inhibition of Staphylococcus Aureus Growth

An in vitro study was conducted to ascertain activity of the fusidic acid composition of Example 1 (2% fusidic acid) against Methicillin resistant (MRSA) Staphylococcus aureus by product inhibition assay and to determine the minimum inhibition concentrations (MIC 50/MIC 90) of mupirocin and fusidic acid against Methicillin resistant Staphylococcus aureus by an agar dilution method.

For the product inhibition assay, Mueller Hinton Agar plates were seeded with a standard MRSA strain (ATCC 43300) or one of the four clinical strains identified in Table 6-1. Individual 8 mm wells were cut in the agar plates and the plugs were removed. 100 μl of the fusidic acid composition was applied into the wells in duplicate. The inoculated plates were incubated aerobically for 24 hours at 37° C. Zones of inhibition by the fusidic acid composition against the test strains were determined. The results of this analysis are shown in Table 6-1.

TABLE 6-1
Zones of Inhibition Against Methicillin Resistant S. aureus.

| | | Mean Zone of Inhibition for MRSA Test | | | | | |
|---|---|---|---|---|---|---|---|
| | | Strains (mm) | | | | | |
| | | Standard | | | | | Mean Zone |
| | | Strain | Clinical Strains | | | | of Inhibition |
| Topical | | ATCC | No. | No. | No. | No. | of all Strains |
| Antimicrobial | Conc. | 43300 | 4287 | 4339 | 4371 | 4378 | (mm) |
| Fusidic | 2% | 36 | 33 | 35 | 32 | 31 | 33.4 |
| acid cream | | | | | | | |
| (Example 1) | | | | | | | |

As shown in Table 6-1, the 2% fusidic acid cream of Example 1 was found to have biological activity against all of the tested MRSA strains, with demonstrably large zones of inhibition (mean=33.4 mm).

Minimum inhibitory concentrations of mupirocin and the fusidic acid against the test strains in Table 6-1 were determined by an agar dilution method on Mueller Hinton agar following Clinical Laboratory Standards Institutes (CLSI) guidelines. The results of this analysis are shown in Table 6-2.

TABLE 6-2
Minimum inhibitory concentrations of mupirocin and fusidic acid

| | Minimum Inhibitory | Minimum Inhibitory |
|---|---|---|
| | Concentration | Concentration |
| Antimicrobial Compound | (MIC50, μg/mL) | (MIC90, μg/mL) |
| Mupirocin ointment | 2 | 4 |
| Fusidic acid, | 0.25 | 0.25 |
| Example 1 cream | | |

As shown in Table 6-2, the MIC 50 of Mupirocin ointment and the fusidic acid cream of Example 1 were, respectively, 2 μg/mL and 0.25 μg/mL. The MIC 90 of Mupirocin and the fusidic acid cream of Example 1 were, respectively 4 μg/mL and 0.25 μg/mL. All five isolates including standard strain and clinical isolates were found sensitive to fusidic acid and mupirocin by agar dilution.

In a further experiment, a product inhibition assay was performed comparing 2% fusidic acid cream of Example 1 and Fucidin™ cream (Leo Pharmaceutical, Denmark) against both methicillin sensitive (MSSA) and methicillin resistant (MRSA) S. aureus strains. The results of this experiment are shown in Table 6-3.

TABLE 6-3
Zones of Inhibition by fusidic acid cream of Example 1 and
Fucidin ™ Cream Against Methicillin Sensitive
(MSSA) and Methicillin Resistant (MRSA) Strains of S. aureus

| | Topical Antimicrobial Compositions | |
|---|---|---|
| | Fusidic acid composition | Fucidin ™ cream |
| | (Example 1) | (Leo-Ranbaxy) |
| Staphylococcus aureus | Zone Diameter (mm) | |
| ATCC 25923 (MSSA) | 25 | 28.33 |
| ATCC 29213 (MSSA) | 26 | 30 |
| ATCC 43300 (MRSA) | 25 | 28 |
| ATCC BAA-1680 (MRSA) | 27 | 30 |
| ATCC BAA-1688 (MRSA) | 29 | 28 |
| ATCC BAA-1689 (MRSA) | 25 | 31 |
| ATCC BAA-1690 (MRSA) | 29.3 | 32 |
| ATCC BAA-1694 (MRSA) | 30 | 33.6 |
| ATCC BAA-1696 (MRSA) | 26 | 30 |
| ATCC BAA-1697 (MRSA) | 26 | 30 |
| ATCC BAA-1698 (MRSA) | 30 | 28 |
| ATCC BAA-1700 (MRSA) | 25 | 28 |
| ATCC BAA-1701 (MRSA) | 26 | 28.33 |

The results in Table 6-1 to 6-3 highlight the potency of fusidic acid in the composition of Example 1 compared to antimicrobials in other topical products.

### Example 7

### Reduction in Likelihood of Creating or Developing Antibiotic Resistant Mutants

An In Vitro Study was Conducted to (1) Determine the Minimum Inhibition concentrations (MIC) of 2% fusidic acid cream (Example 1), Fucidin™ cream, and T-Bact™ ointment against seven strains Staphylococcus aureus, including (ATCC 25923, ATCC 43300, ATCC 700699, 2 clinical isolates of methicillin sensitive S. aureus (MSSA) and 2 clinical isolates of methicillin resistant S. aureus (MRSA) and (2) screen for the development of resistant mutants of Staphylococcus aureus (all the seven strains) by sub-MIC exposure to 2% fusidic acid cream of Example 1, Fucidin® cream and T-Bact® ointment (up to 70 serial passages) using Whole Genome Sequencing (WGS).

Minimum inhibitory concentrations for the topical antimicrobial compositions against various MSSA and MRSA S. aureus strains were determined by the product inhibition assay described in Example 6. The results of this assay are shown in Table 7-1 below.

To evaluate resistant mutant development following serial passages under sub-MIC exposure, bacterial suspensions of S. aureus (ATCC 25923, ATCC 43300, ATCC 700699), 2 clinical MSSA isolates and 2 clinical MRSA isolates were plated in 96-well microtiter plates exposed to sub-MIC concentrations of methicillin for 6 hours at 37° C. The MICs were determined as described in Example 6. This process of sub lethal concentration exposures and MIC determinations were carried out for 70 passages and were assessed for increases in MIC values at each passage. Whole Genome Sequencing (WGA) analysis was carried out with DNA extracted from the reference strains and strains showing higher MIC values following passage using a DNA minikit (QIAGEN, Hilden Germany). The DNA was sent to Medgenome Lab Bengaluru for genome analysis. The increases in MIC values are summarized in Table 7-2.

TABLE 7-1
Minimum Inhibitory Concentrations for Compositions Against Various
MSSA and MRSA S. aureus Strains and Clinical Isolates

| | 2% fusidic acid | | |
|---|---|---|---|
| | cream (Example 1) | Fucidin ™ | T-Bact ™ |
| Strain | Minimum inhibitory concentration (μg) | | |
| S. aureus ATCC 700699 | 16 | 16 | 16 |
| (MRSA) | | | |
| S. aureus ATCC 43300 | 16 | 64 | 16 |
| (MRSA) | | | |
| S. aureus ATCC 25923 | 16 | 64 | 32 |
| (MSSA) | | | |
| S. aureus Clinical Isolate 1 | 16 | 16 | 16 |
| (MSSA) | | | |
| S. aureus Clinical Isolate 2 | 16 | 64 | 32 |
| (MSSA) | | | |
| S. aureus Clinical Isolate 3 | 32 | 64 | 16 |
| (MRSA) | | | |
| S. aureus Clinical Isolate 4 | 32 | 64 | 16 |
| (MRSA) | | | |

TABLE 7-2
Resistant Mutant Development By 50 Serial Passages to Sub MIC Exposure

| | 2% fusidic acid | | |
|---|---|---|---|
| | cream (Example 1) | Fucidin ™ | T-Bact ™ |
| Strain | Minimum inhibitory concentration (μg) | | |
| S. aureus ATCC 700699 | 16 to passage 58; | 16 to passage 35; | 16 to passage 60; |
| (MRSA) | 32 to passage 70 | 64 to passage 56; | 32 to passage 70 |
| | | 128 to passage 70 | |
| S. aureus ATCC 43300 | 16 to passage 66; | 64 to passage 56; | 16 to passage 58; |
| (MRSA) | 32 to passage 70 | 128 to passage 70 | 32 to passage 70 |
| S. aureus ATCC 25923 | 16 to passage 61; | 16 to passage 56; | 32 to passage 21; |
| (MSSA) | 32 to passage 70 | 128 to passage 70 | 64 to passage 68; |
| | | | 128 to passage 70 |
| S. aureus Clinical Isolate 1 | 16 | 16 to passage 35; | 16 to passage 65; |
| (MRSA) | | 64 to passage 56; | 32 to passage 70 |
| | | 128 to passage 70 | |
| S. aureus Clinical Isolate 2 | 16 to passage 47; | 64 | 32 to passage 11; |
| (MRSA) | 32 to passage 70 | | 64 to passage 70 |
| S. aureus Clinical Isolate 3 | 32 | 64 | 16 to passage 47; |
| (MRSA) | | | 32 to passage 62; |
| | | | 128 to passage 70 |
| S. aureus Clinical Isolate 4 | 32 | 64 to passage 61; | 16 to passage 17; |
| (MRSA) | | 128 to passage 70 | 64 to passage 66; |
| | | | 128 to passage 70 |

TABLE 7-3
Resistant Mutant Development

| | 2% fusidic acid | | |
|---|---|---|---|
| | cream (Example 1) | Fucidin ™ | T-Bact ™ |
| Strain | Minimum inhibitory concentration (μg) | | |
| S. aureus ATCC 700699 | One-fold rise | Four-fold rise | One-fold rise |
| (MRSA) | | | |
| S. aureus ATCC 43300 | One-fold rise | One-fold rise | One-fold rise |
| (MRSA) | | | |
| S. aureus ATCC 25923 | One-fold rise | One-fold rise | Two-fold rise |
| (MSSA) | | | |
| S. aureus Clinical Isolate 1 | No change | Four-fold rise | One-fold rise |
| (MRSA) | | | |
| S. aureus Clinical Isolate 2 | One-fold rise | No change | One-fold rise |
| (MRSA) | | | |
| S. aureus Clinical Isolate 3 | No change | No change | One-fold rise |
| (MRSA) | | | |
| S. aureus Clinical Isolate 4 | No change | One-fold rise | Four-fold rise |
| (MRSA) | | | |

### 2% Fusidic Acid Cream (Example 1) Treatment

The 2% fusidic acid cream (Example 1) showed the MIC value of 16 μg for MRSA standard strains ATCC 43300, ATCC700699, MSSA standard strain ATCC 25923, MSSA clinical isolates and 32 μg for clinical isolates of MRSA (Table 7-1). As shown in Tables 7-2 and 7-3, when all the strains were subjected to sub-MIC concentration of respective MIC values, ATCC700699 strain showed one-fold rise in MIC by 59th passage, strain ATCC 43300 by 67th passage, ATCC 25923 by 62nd passage and one of the MSSA clinical isolate by 48th passage as shown in Table 6-2. The MRSA clinical isolates and one of the MSSA clinical isolates did not develop any resistant mutants until 70 passages.

The WGS analysis of the S. aureus strains with 2% fusidic acid cream (Example 1) treatment revealed 150 genetic mutations or variations in the MSSA (ATCC 25923) reference strain and 84 mutations in the MRSA (ATCC 700699) reference strain. S. aureus (ATCC 43300) and MSSA clinical isolate 1 showed an increase in MIC values, but no significant genetic mutations were documented.

### Fucidin Cream Treatment

The Fucidin™ cream showed a MIC of 16 μg for MRSA standard strain ATCC 700699 and one of the clinical isolates of MSSA and a MIC value of 64 μg for the rest of the isolates (Table 7-1). As shown in Tables 7-2 and 7-3, when all the strains were subjected to sub-MIC concentrations of methicillin, a two-fold rise in MIC was observed in the MRSA standard strain ATCC700699 and in one of the MSSA clinical isolates (From 16 μg to 64 μg). ATCC 43300 and ATCC 25923 showed a one-fold rise in the MIC by the 57th passage and one of the MRSA clinical strains showed one-fold rise in MIC by the 62nd passage. One MSSA and one MRSA clinical isolate did not develop resistant mutants, even up to 70 passages.

The WGS analysis of the S. aureus strains with Fucidin cream treatment revealed 185 genetic mutations in one of the clinical MSSA isolates; 102 genetic mutations in the MRSA standard strain ATCC 700699; 95 genetic mutations in the MRSA standard strain ATCC 43300; 85 genetic mutations in the MSSA standard strain ATCC 25923; and 76 genetic mutations in one of the clinical MRSA isolates of MRSA.

### T-Bact™ Ointment Treatment

The T-Bact™ ointment showed the MIC of 16 μg for MRSA standard strain ATCC700699, Staph aureus ATCC 43300, one of the clinical isolates of MSSA, MRSA clinical isolates and MIC value was 32 μg for S. aureus ATCC 25923 and one clinical isolate.

As shown in Tables 7-2 and 7-3, when all the strains were subjected to sub-MIC concentration of respective MIC values, all the seven isolates developed resistant mutants. One-fold rises in MIC were observed in the MRSA standard strain ATCC700699 by the 61st passage; in the MRSA standard strain ATCC 43300 by the 59th passage; in one of the MSSA clinical isolates by the 66th passage; and in another by the 12th passage. ATCC 25923 strain showed a one-fold rise by the 22nd passage and another one-fold rise by the 69th passage (32 to 64 to 128 μg), One of the MRSA strains showed a two-fold rise in MIC by the 63rd passage (32 by 48th passage and 64 by the 63rd passage) and another MRSA stain showed a four-fold rise in MIC by the 67th passage (64 by the 18th passage and 128 by the 67th passage) as shown in Tables 7-2 and 7-3.

WGS analysis of the strains with T-Bact™ ointment treatment, it was noted that 158 mutations or genetic variations with the MSSA standard strain ATCC 25923 and one of the clinical isolates of MSSA, followed by 150 variations for ATCC 700699, 115 with ATCC 43300 and variations ranged from 89 to 85 with another MSSA clinical isolate and two MRSA clinical isolates. The WGS analysis of the resistant strains did not show mutations in any of the resistant genes, fus A, fus B, fus C, fus D or mup R and showed genetic variations (SNPs) and mutations at the chromosomal level as discussed.

Taken together, 2% fusidic acid cream of Example 1 showed low resistance potential in vitro compared to Fucidin™ whereas T-Bact™ ointment had more resistance development potential. When the WGS analysis of the strains showing raise in MIC were analyzed, even though major mutants in target genes were not observed whereas variations and mutations were noted in chromosomal level. Strains exposed to T-Bact™ showed the most genetic variation followed by Fucidin™ cream and then by 2% fusidic acid cream of Example 1. Thus, the study showed that 2% fusidic acid cream of Example 1 was comparatively better than Fucidin™ cream and T-Bact™ phenotypically and genotypically and is less likely to support the development of resistant mutants when used topically, even up to 8 weeks.

### Example 8

Method of Treating Wounds and Ameliorating Associated Itch and/or Pain

A meta-analysis of randomized controls trials of 2% fusidic acid cream (Example 1) vs. Fucidin™ cream (Leo-Ranbaxy), SOFRAMYCIN® cream (Sanofi-Aventis) and Bactroban™ cream (T-Bact™ in India, GlaxoSmithKline Pharmaceuticals (GSK)) in skin infection conditions was carried out. A principal objective was to compare the efficacy, safety and tolerability of fusidic acid with biopolymer (chitosan) vs. the foregoing reference product without biopolymer among all patients undergoing different trials conducted at Quest Life Sciences, Chennai, India and secondarily between patients in each study category in the studies conducted at Quest Life Sciences.

The trials compared: (i) 2% fusidic acid cream of Example 1 (fusidic acid 2% w/w) vs. Fucidin™ 2% w/w cream (Leo-Ranbaxy); (ii) 2% fusidic acid cream of Example 1 cream vs. SOFRAMYCIN 1% w/w cream (Sanofi-Aventis); and (iii) 2% fusidic acid cream of Example 1 vs. Bactroban™ 2% w/w cream (GSK).

Various scales and parameters were evaluated in these studies, including:
(1) Visual Analogue Scale (VAS), which was done for the assessment of pruritus by the patient. It ranges from 1 to 10, 1 being the lowest and 10 the maximum. Visual analogue scale: 1-2-3-4-5-6-7-8-9-10 for pruritus.
(2) Physician Global Evaluation Score (PGES), which representing an overall evaluation summarized on a five-point scale on visit 1, visit 2 and visit 3, where a score of 5 was “excellent”; a score of 4 was “good”; a score of 3 was “no change”; a score of 2 was “poor”, and a score of 1 was “worse”.
(3) Patient's Compliance, where patients were asked for itching/irritation/pain in the wound area on visit 1, visit 2 and visit 3, where a score was given based on the opinion of the patients and where 0=absent; 2=moderate; and 3=severe.
(4) Percentage Wound Contraction (PWC), which was calculated after visit 1, visit 2, and visit 3 using the formula where:

$$Percentage of wound contraction = \frac{Initial wound size - Specified day wound size}{Initial wound size} \times 100$$

(5) Wound Re-Epithelialization Score (WRS), which was measured and recorded visit 1, visit 2, and visit 3, where a score of 1=“very slow”; a score of 2=“slow”; a score of 3=“moderate”; and a score of 4=“rapid”.

In the above studies, the baseline and visit 3 data of each scale and differences between the baseline and visit 3 scores were recorded with respect to their corresponding groups in addition to the baseline and visit 3 scores. Using SPSS 16.0, normally distributed data were expressed as Mean±Standard error of mean, Standard deviation, 95% and 99% Confidence interval of mean and analyzed by one way analysis of variance (ANOVA). The non-uniform data were expressed as Median and Quartile (Q1, Q3) and further data analysis was done by non-parametric 2 independent samples test followed by Mann-Whitney U test. P value less than 0.05 was considered as statistically significant. The results from these analyses are shown in Tables 8-1 to 8-5 below, where ^{S} refers to products without polymer; N=sample size; SEM=Standard Error of Mean; SD=Standard Deviation; S=Significant; and NS=Not significant.

TABLE 8-1A
Visual Analogue Scale (VAS) - Visit 3 score

| | | | | 95% | P value |
|---|---|---|---|---|---|
| | | | | Confidence | Significance |
| Dose | N | Mean ± SEM | ±SD | Interval | (S) |
| I - Products | 40 | 5.85 ± 0.24 | ±1.52 | (5.36, 6.33) | 0.001^{$} - S |
| with | | | | | |
| Biopolymer | | | | | |
| II - Products | 40 | 4.65 ± 0.23 | ±1.47 | (4.17, 5.12) | — |
| without | | | | | |
| Biopolymer | | | | | |

TABLE 8-1B
Visual Analogue Scale (VAS) - Visit 3 score

| | | | 99% Confidence |
|---|---|---|---|
| Dose | N | Mean | Interval |
| I - Products with | 40 | 5.85 | (5.19, 6.50) |
| Biopolymer | | | |
| II - Products without | 40 | 4.65 | (4.01, 5.28) |
| Biopolymer | | | |

TABLE 8-2A
Patient Compliance Score (PCS) - Visit 3 score

| | | | | 95% | P value |
|---|---|---|---|---|---|
| | | | | Confidence | Significance |
| Dose | N | Mean ± SEM | ±SD | Interval | S or NS |
| I - Products | 40 | 2.65 ± 0.24 | ±0.08 | (2.47, 2.82) | 0.06 - NS |
| with | | | | | |
| Biopolymer | | | | | |
| II - Products | 40 | 2.35 ± 0.23 | ±0.13 | (2.07, 2.62) | — |
| without | | | | | |
| Biopolymer | | | | | |

TABLE 8-2B
Patient Compliance Score (PCS) - Visit 3 score

| | | | 99% Confidence |
|---|---|---|---|
| Dose | N | Mean | Interval |
| I - Products with | 40 | 2.65 | (2.42, 2.87) |
| Biopolymer | | | |
| II - Products without | 40 | 2.35 | (1.98, 2.71) |
| Biopolymer | | | |

TABLE 8-3A
Physician Global Evaluation Score
(PGES) - Visit 3 score - Baseline score

| | | | | 95% | P value |
|---|---|---|---|---|---|
| | | | | Confidence | Significance |
| Dose | N | Mean ± SEM | ±SD | Interval | S or NS |
| I - Products | 40 | 3.75 ± 0.07 | ±0.49 | (3.59, 3.90) | 0.001 - S |
| with | | | | | |
| Biopolymer | | | | | |
| II - Products | 40 | 3.22 ± 0.13 | ±0.83 | (2.95, 3.49) | — |
| without | | | | | |
| Biopolymer | | | | | |

TABLE 8-3B
Physician Global Evaluation Score
(PGES) - Visit 3 score - Baseline score

| | | | 99% Confidence |
|---|---|---|---|
| Dose | N | Mean | Interval |
| I - Products with | 40 | 3.75 | (3.53, 3.96) |
| Biopolymer | | | |
| II - Products without | 40 | 3.22 | (2.86, 3.58) |
| Biopolymer | | | |

TABLE 8-4A
Percentage of Wound Contraction (PWC) - Visit 3% - Baseline %

| | | | | 95% | P value |
|---|---|---|---|---|---|
| | | | | Confidence | Significance |
| Dose | N | Mean ± SEM | ±SD | Interval | S or NS |
| I - Products with | 39 | 99.61 ± 1.02 | ±6.41 | (94.53, 98.69) | <0.001 - S |
| Biopolymer | | | | | (0.000) |
| II - Products | 39 | 77.97 ± 4.40 | ±27.47 | (69.06, 86.88) | — |
| without Biopolymer | | | | | |

TABLE 8-4B
Percentage of Wound Contraction (PWC) - Visit 3% - Baseline %

| | | | 99% |
|---|---|---|---|
| | | | Confidence |
| Dose | N | Mean | Interval |
| I - Products with | 39 | 99.61 | (93.82, 99.40) |
| Biopolymer | | | |
| II - Products without | 39 | 77.97 | (66.04, 89.90) |
| Biopolymer | | | |

TABLE 8-5A
Wound Re-Epithelialization Score
(WRS) - Visit 3 score - Baseline score

| | | | | 95% | P value |
|---|---|---|---|---|---|
| | | Mean ± | | Confidence | Significance |
| Dose | N | SEM | ±SD | Interval | S or NS |
| I - Products with | 40 | 2.87 ± 0.05 | ±0.33 | (2.76, 2.98) | 0.002 - S |
| Biopolymer | | | | | |
| II - Products | 39 | 2.38 ± 0.14 | ±0.90 | (2.09, 2.67) | — |
| without | | | | | |
| Biopolymer | | | | | |

TABLE 8-5B
Wound Re-Epithelialization Score
(WRS) - Visit 3 score - Baseline score

| | | | 99% |
|---|---|---|---|
| | | | Confidence |
| Dose | N | Mean | Interval |
| I - Products with | 40 | 2.87 | (2.73, 3.01) |
| Biopolymer | | | |
| II - Products without | 39 | 2.38 | (1.99, 2.77) |
| Biopolymer | | | |

Based on the results, the products with biopolymer were found provide highly significant benefits to treated subjects with respect to Visual analogue scale, Physician global evaluation score, Percentage wound contraction and Wound re-epithelization score in comparison with subjects treated with reference products without biopolymer. At the end of treatment, bacterial culture examination reports were negative in 100% of patients who had been treated with the products with biopolymer and negative in 95% of patients who had been treated with the reference products without biopolymer.

The results of the meta-analysis demonstrate that the fusidic acid composition with the biopolymer (chitosan), i.e., 2% fusidic acid cream of Example 1, was more effective than the reference products without biopolymer in achieving clinical improvement or resolution of skin infections. This is reflected in better safety and efficacy profiles and compliance with treatments compared to reference products without biopolymer and may achieve better compliance with treatment. Inclusion of patients in these studies was based on clinical diagnosis, making its findings directly applicable to routine clinical practice. These clinical trials show that the fusidic acid composition in a chitosan base provides superior patient centric therapeutic benefits over the other products tested.

### Example 9

### Method of Treating Wounds

Wound healing of four test compositions including placebo was evaluated in a full thickness, porcine wound model. The test compositions included (1) the 2% fusidic acid cream with chitosan biopolymer (Example 1), (2) chitosan cream, (3) fusidic acid cream, and (4) a placebo cream. Table 9-1 provides details of the compositions.

TABLE 9-1
Compositions for Wound Healing

| | | | Compo- |
|---|---|---|---|
| Composition | | Composition | sition |
| Identification | Active Ingredients | Appearance | pH |
| 1 | sodium fusidate, chitosan, nitric | White cream | 4.0 |
| | oxide and lactic acid (Example 1) | | |
| 2 | chitosan, lactic acid | White cream | 4.0 |
| 3 | sodium fusidate, nitric acid | White cream | 4.0 |
| 4 | None (placebo) | White cream | 4.0 |

One animal (body weight 40.23 kg) was taken for this study, and on day 0 of the study 24 full thickness wounds (2.5×2.5 cm²˜6-10 mm deep) cubic cm (square shaped) were created, 12 wounds each side of the animal. The wounds were created with a scalpel blade (Number 11) after properly marking the wound areas with a marker. A baseline planimetry (L1×B1=A1, L2×B2=A2, Mean Area=A1+A2/2) was done for each wound area measurement.

Each test composition was applied to six individual wounds in an amount so that the composition was in direct contact to the wound site. The wounds were then covered with sterile non adherent dressing, followed by cling wrap, vet wrap and elasticon. Each test composition was applied at least twice a week over the 28 day study, on days 3, 7, 10, 14, 18, 21 and 24. Wound healing was assessed by planimetry and quantitative assessment of wound scarring with photography on 7, 14, 21 and 28 days. Biopsy samples of one wound for each test composition were taken on days 7, 14, 21 and 28 for histopathology. Scoring systems are set forth in Tables 9-2 and 9-3. In Table 9-2, a lower Visual Analogue score for biofilm detection reflects better healing. In Table 9-3, a lower score indicates better wound healing. Results are in Tables 9-4, 9-5, 9-6 and 9-7.

TABLE 9-2
Visual Analogue Wound Scarring Score Criterion and Score

| | | | |
|---|---|---|---|
| A | Color (compared | Perfect | 1 |
| | to surrounding | Slight mismatch | 2 |
| | skin) | Obvious mismatch | 3 |
| | | Gross mismatch | 4 |
| B | Surface | Matte | 1 |
| | | Shiny | 2 |
| C | Contour | Flush with surrounding skin | 1 |
| | | Slightly Proud/Indented | 2 |
| | | Hypertrophic | 3 |
| | | Keloid | 4 |
| D | Distortion | None | 1 |
| | | Mild | 2 |
| | | Moderate | 3 |
| | | Severe | 4 |
| E | Texture | Normal | 1 |
| | | Just Palpable | 2 |
| | | Firm | 3 |
| | | Hard | 4 |
| F | Visual Analogue | Scanty | 1 |
| | score for biofilm | Focal | 2 |
| | detection | Diffuse | 3 |
| | | Throughout | 4 |

TABLE 9-3
Histopathology Criterion and Score
Epidermis

| | |
|---|---|
| None | 0 |
| Mild epithelialisation | 1 |
| Epithelialisation of more than half of wound | 2 |
| Complete epithelialisation | 3 |
| (Note: A higher score indicates better wound healing) | |

Epidermis

| | |
|---|---|
| Normal | 0 |
| Some restoration of rete ridges | 1 |
| No restoration of rete ridges | 2 |

Dermis

| | | |
|---|---|---|
| Collagen | Normal basket weave pattern | 0 |
| orientation | <25% abnormal | 1 |
| | 26-50% abnormal | 2 |
| | 51-75% abnormal | 3 |
| | 76-100% abnormal | 4 |
| | Keloid-like fiber orientation | 5 |
| Collagen fiber | Normal fiber bundle density | 0 |
| Density | <25% abnormal | 1 |
| | 26-50% abnormal | 2 |
| | 51-75% abnormal | 3 |
| | 76-100% abnormal | 4 |
| | Keloid-like fibers | 5 |
| Collagen fiber | Normal fiber maturity | 0 |
| maturity | <25% abnormal | 1 |
| | 26-50% abnormal | 2 |
| | 51-75% abnormal | 3 |
| | 76-100% abnormal | 4 |
| | Keloid-like fibers | 5 |

TABLE 9-4
Planimetry Data

| Study | Mean Wound size (cm²) ± SD | | | |
|---|---|---|---|---|
| Test | 1 (Fusidic acid + | 2 | 3 | 4 |
| Day | chitosan) | (Chitosan) | (Fusidic acid) | (Placebo) |
| 0 | 6.25 ± 0 | 6.25 ± 0 | 6.25 ± 0 | 6.25 ± 0 |
| 7 | 8.38 ± 0.15 | 8.45 ± 0.06 | 8.57 ± 0.27 | 8.53 ± 0.22 |
| 14 | 5.45 ± 0.21 | 5.78 ± 0.06 | 6.23 ± 0.21 | 6.66 ± 0.19 |
| 21 | 1.22 ± 0.11 | 1.44 ± 0.10 | 1.58 ± 0.14 | 1.99 ± 0.09 |
| 28 | 0.10 ± 0.01 | 0.17 ± 0.02 | 0.32 ± 0.04 | 0.55 ± 0.13 |

Composition 1 showed mean area of 8.38±0.15 cm² on day 7 that reduced to 0.10±0.01 cm² on day 28. Composition 2 showed wound reduction from 8.45±0.06 cm² to 0.17±0.02 cm². Composition 3 showed wound reduction from 8.57±0.27 cm² to 0.32±0.04 cm². Composition 4 showed wound reduction from 8.53±0.22 cm² to 0.55±0.13 cm². There was no scarring found in all the wounds. Wound healing score was better in Composition and Composition 2 as compared to Composition 3 and Composition 4, with a rate of wound healing of each composition in order Composition 1>Composition 2>Composition 3>Composition 4.

TABLE 9-5
Wound Scar Score

| Study | Wound Scar Score (see Table 9-2 for scoring system) | | | |
|---|---|---|---|---|
| Test | 1 (Fusidic acid + | 2 | 3 (Fusidic | 4 |
| Day | chitosan) | (Chitosan) | acid) | (Placebo) |
| 0 | 13.00 ± 0.00 | 13.00 ± 0.00 | 13.00 ± 0.00 | 13.00 ± 0.00 |
| 7 | 13.50 ± 0.55 | 13.50 ± 0.55 | 13.50 ± 0.55 | 13.50 ± 0.84 |
| 14 | 11.00 ± 0.71 | 11.20 ± 0.84 | 12.80 ± 0.84 | 14.80 ± 1.10 |
| 21 | 8.00 ± 0.00 | 8.50 ± 0.58 | 9.25 ± 1.89 | 12.75 ± 0.96 |
| 28 | 6.00 ± 0.00 | 6.00 ± 0.00 | 6.33 ± 0.58 | 10.33 ± 1.15 |

TABLE 9-6
Epidermis and Dermis Histopathological Score

| | | | Epidermis | Dermis | | | | | | | |
|---|---|---|---|---|---|---|---|---|---|---|---|
| Treatment | | | Restoration | Collagen | Collagen | Collagen | | | | | |
| (Composition | Wound | Wound | of rete | fibre | fibre | fibre | Neovascu- | Inflam- | | | |
| No.) | Duration | No. | ridges | Orientation | Density | Maturity | larisation | mation | Scab | Total | Mean |
| 1 | Day 7 | W2 | 2 | 4 | 4 | 4 | 4 | 3 | 3 | 24 | 24 |
| | Day 14 | W4 | 2 | 3 | 3 | 3 | 3 | 2 | 2 | 18 | 18 |
| | Day 21 | W5 | 1 | 1 | 1 | 1 | 2 | 1 | 1 | 8 | 8 |
| | Day 28 | W1 | 1 | 2 | 2 | 2 | 2 | 0 | 0 | 9 | 7.00 |
| | Day 28 | W3 | 0 | 1 | 1 | 1 | 2 | 1 | 0 | 6 | |
| | Day 28 | W6 | 1 | 1 | 1 | 1 | 2 | 0 | 0 | 6 | |
| 2 | Day 7 | W9 | 2 | 4 | 4 | 4 | 4 | 3 | 3 | 24 | 24 |
| | Day 14 | W7 | 2 | 3 | 3 | 3 | 4 | 2 | 3 | 20 | 20 |
| | Day 21 | W12 | 1 | 2 | 2 | 2 | 2 | 1 | 1 | 11 | 11 |
| | Day 28 | W8 | 0 | 2 | 2 | 1 | 2 | 1 | 0 | 8 | 6.33 |
| | Day 28 | W10 | 0 | 1 | 1 | 1 | 2 | 0 | 0 | 5 | |
| | Day 28 | W11 | 0 | 1 | 2 | 1 | 2 | 0 | 0 | 6 | |
| 3 | Day 7 | W16 | 2 | 4 | 4 | 4 | 4 | 4 | 3 | 25 | 25 |
| | Day 14 | W14 | 1 | 3 | 3 | 3 | 2 | 2 | 1 | 15 | 15 |
| | Day 21 | W15 | 1 | 2 | 2 | 2 | 2 | 1 | 1 | 11 | 11 |
| | Day 28 | W13 | 1 | 2 | 2 | 2 | 2 | 1 | 0 | 10 | 10.33 |
| | Day 28 | W17 | 1 | 2 | 2 | 2 | 3 | 1 | 0 | 11 | |
| | Day 28 | W18 | 1 | 2 | 2 | 2 | 3 | 0 | 0 | 10 | |
| 4 | Day 7 | W19 | 2 | 4 | 4 | 4 | 3 | 4 | 3 | 24 | 24 |
| | Day 14 | W21 | 2 | 3 | 2 | 2 | 2 | 3 | 2 | 16 | 16 |
| | Day 21 | W22 | 1 | 2 | 2 | 2 | 4 | 2 | 2 | 15 | 15 |
| | Day 28 | W20 | 2 | 2 | 2 | 2 | 4 | 1 | 0 | 13 | 11.33 |
| | Day 28 | W23 | 1 | 2 | 2 | 2 | 3 | 1 | 0 | 11 | |
| | Day 28 | W24 | 1 | 2 | 2 | 1 | 3 | 1 | 0 | 10 | |

TABLE 9-7
Epidermis Epithelialization Score

| Treatment | | | | |
|---|---|---|---|---|
| (Composition | Wound | | Epidermis | |
| No.) | Duration | Wound No. | Epithelialisation | Mean |
| 1 | Day 7 | W2 | 1 | 1 |
| | Day 14 | W4 | 1 | 1 |
| | Day 21 | W5 | 3 | 3 |
| | Day 28 | W1 | 3 | 3.00 |
| | Day 28 | W3 | 3 | |
| | Day 28 | W6 | 3 | |
| 2 | Day 7 | W9 | 1 | 1 |
| | Day 14 | W7 | 1 | 1 |
| | Day 21 | W12 | 3 | 3 |
| | Day 28 | W8 | 3 | 3.00 |
| | Day 28 | W10 | 3 | |
| | Day 28 | W11 | 3 | |
| 3 | Day 7 | W16 | 1 | 1 |
| | Day 14 | W14 | 1 | 1 |
| | Day 21 | W15 | 3 | 3 |
| | Day 28 | W13 | 3 | 2.67 |
| | Day 28 | W17 | 2 | |
| | Day 28 | W18 | 3 | |
| 4 | Day 7 | W19 | 1 | 1 |
| | Day 14 | W21 | 1 | 1 |
| | Day 21 | W22 | 3 | 3 |
| | Day 28 | W20 | 3 | 3.00 |
| | Day 28 | W23 | 3 | |
| | Day 28 | W24 | 3 | |

Epithelialisation (neoepithelialisation) is characterized by formation of epidermis in the covering of dermis by newly formed epithelium from days 21 to 28 in animals treated with one of the compositions with active ingredient (Compositions 1, 2 and 3). Lower mean histopathological score of restoration of rete ridges, orientation, density, and maturity of collagen fiber; angiogenesis (neovascularization), scab formation and inflammation was observed in treatment group compared to control group from Day 21 to Day 28. These results indicate earlier, and faster healing of animals treated with one of the compositions with active ingredient (Compositions 1, 2 and 3) compared to animals treated with the control, placebo composition (Composition 4). In dermis, wound healing was indicated by horizontal orientation, fascicle pattern of fibers, maturation of collagen and reduction in stromal components, as the days progressed from 21 to 28 of treatment group as compared to control.

### Example 10

### Inhibition of Staphylococcus Aureus Growth

An in vitro study was to evaluate the wound healing and antibacterial potential of four test compositions: (1) the 2% fusidic acid cream with chitosan biopolymer (Example 1), (2) chitosan cream, (3) fusidic acid cream, and (4) a placebo cream. The ingredients in each test composition are set forth in Table 9-1.

Eight animals of species Susscrofa, mixed breed, male, with a weight between 50.1-69.7 kg and an age of 8-12 weeks were obtained. Animals were healthy and observed daily for signs of illness or distress. Feed and water were withdrawn over-night prior to every procedural day; food was withheld further for a period of 6 hours after the procedure for the surviving animals. Seven days prior to application of the test compositions, study animals were weighed, induced for anesthesia with 15 mg/kg ketamine IM, 2.5 mg/kg xylazine IM and 0.05 mg/kg atropine followed by 1-3% isoflurane inhalation anesthesia through face mask. A total 12 wounds (0.75-1.5 mm deep and 6.0×6.0 cm², 6 on each side) were created on the back of the animal with the help of dermatome (SOP/PBS/PAT/055). One mL bacterial inoculum (109 CFU/mL, S. aureus) per wound site was applied. Sterile gauze, waterproof dressing and bandage was applied in each wound site. Each animal work a waterproof jacket was worn to protect the dressing.

Seven days after wound creation, the animals were prepared under proper analgesia and anesthesia for application of the test compositions. The dressings from the wounds were removed, sample was collected in sterile swabs tubes with media for the confirmation of biofilm, and wound measurements were done. The test compositions were then applied onto the wound (test day zero). On test days 7, 14, 21 and 28 dressings were removed from all the wounds. Samples for colony count, gross inspection wound score, wound measurement and photography were taken. Biopsy samples from wound for immunohistopathology, histopathology, colony count and collagen were also taken. At each time point, two animals were sacrificed for biopsies, for wound skin sample collections, necropsy observation and histology. For animals continuing to the next study time point, the test composition was reapplied to the wound and a fresh dressing with sterile gauze and waterproof covering was applied.

The method for biofilm detection from the wound swab taken on test day zero was as follows. The sample obtained from each wound on the sterile swab was sub-cultured on blood agar or nutrient agar. A single colony from each sub-cultured plate was inoculated in a glass tube containing tryptone soya broth. The tubes to be incubated overnight at 37° C. under aerobic conditions. A 200 μL aliquot from each of the inoculated tryptone soya broth tubes was aseptically transferred in the wells of a flat bottomed micro-well plastic plate. The inoculated micro-well plastic plate was incubated overnight at 37±1° C. without sealing of the plate for proper oxygenation. Next day, the contents to be discarded by inverting the plate and striking it on filter paper. The micro-well plastic plate was washed with 200 μL phosphate buffer solution (pH 7.2) once by adding 200 μL each well and then discarded. A 200 μL of freshly prepared sodium acetate was added to each well (for bio-film fixation) for 10 minutes and then discarded. This was followed by adding 200 μL crystal violet (0.1%) to each well for bio-film staining. The plates were kept at room temperature for 30 minutes, and then the stain to be discarded. The washing step was repeated once more. Finally, the plate was left to dry at room temperature for one hour, after which, the absorbance to be read on a spectrophotometer at 620 nm OD for the confirmation of biofilm. All the wounds were confirmed to develop biofilm with the OD values and visual observation.

All the wounds on the animals were created after marking a square wound of 6 cm×6 cm (˜36 cm²) with 2 runs of dermatome taking out 2 slices per wound of 0.75 mm thickness. For wound measurements starting on test day zero, for planimetry L-shaped paper ruler was used to measure the two adjacent sides of each wound in such a way that two sets of readings were obtained for the length and breadth respectively such as left lateral length (L1), lower breadth (B1), right lateral (L2) and top breadth (B2). There were two sets of areas were obtained such as A1 (L1× B1) and A2 (L2× B2). Each wound area is calculated as mean of A1 and A2.

Data for the planimetry measurement of wound size at test days 7, 14, 21 and 28 are in Table 10-1 and in FIGS. 5A-5E.

TABLE 10-1
Planimetry Data

| | Mean Wound size (cm²) ± SD | | | |
|---|---|---|---|---|
| Study | 1 (Fusidic | | | |
| Test | acid + | 2 | 3 (Fusidic | 4 |
| Day | chitosan) | (Chitosan) | acid) | (Placebo) |
| 0 (P1, P2) | 37.10 ± 18.28 | 36.50 ± 17.63 | 36.60 ± 17.63 | 36.50 ± 17.32 |
| 0 (P3, P4) | 36.50 ± 17.32 | 36.20 ± 17.32 | 36.20 ± 17.32 | 36.60 ± 17.63 |
| 0 (P5, P6) | 36.70 ± 17.96 | 36.40 ± 17.32 | 36.0 ± 17.3 | 36.30 ± 17.3 |
| 0 (P7, P8) | 36.8 ± 18.10 | 36.5 ± 17.49 | 36.35 ± 17.63 | 36.60 ± 17.67 |
| 7¹ | 1.69 ± 0.1 | 4.77 ± 2.33 | 22.22 ± 8.15 | 29.85 ± 16.50 |
| 14² | 0.62 ± 0.40 | 9.59 ± 2.33 | 4.86 ± 1.90 | 27.74 ± 14.83 |
| 21³ | 0.00 ± 0.0 | 0.16 ± 0.0 | 0.0 ± 0.0 | 1.04 ± 0.0 |
| 28⁴ | 0.00 ± 0.0 | 2.0 ± 0.0 | 0.00 ± 0.0 | 0.37 ± 0.0 |

¹Animals P1, P2.
²Animals P3, P4.
³Animals P5, P6.
⁴Animals P7, P8.

The healing quality was assessed using the wound scarring evaluation through visual analogue score (Table 9-2 of Example 9), as per the scoring criteria for color, surface, contour, distortion and texture of the treated wound. A lower sum of the visual analogue score reflects better healing. Results are in Table 10-2 and in FIGS. 6A-6D. It was found that wounds treated with test composition 1 and test composition 3 clearly showed better healing (less sums of the visual analogue score) than wounds treated with test composition 2 and test composition 4 (placebo) until day 14. On test day 21 and test day 28 almost all the treated wounds showed similar sum of the visual analogue score.

TABLE 10-2
Wound Scar Score

| Study | Wound Scar Score (see Table 9-2 for scoring system) | | | |
|---|---|---|---|---|
| Test | 1 (Fusidic acid + | 2 | 3 (Fusidic | 4 |
| Day | chitosan) | (Chitosan) | acid) | (Placebo) |
| 7¹ | 6.00 ± 0.0 | 9.50 ± 0.55 | 11.33 ± 0.51 | 14.00 ± 0.00 |
| 14² | 5.83 ± 0.40 | 8.33 ± 0.81 | 6.66 ± 0.51 | 9.83 ± 1.60 |
| 21³ | 5.00 ± 0.00 | 5.16 ± 0.40 | 5.00 ± 0.00 | 5.33 ± 0.51 |
| 28⁴ | 5.00 ± 0.00 | 5.00 ± 0.00 | 5.00 ± 0.00 | 5.00 ± 0.00 |

¹Animals P1, P2.
²Animals P3, P4.
³Animals P5, P6.
⁴Animals P7, P8.

With regard to the biofilm confirmation on test day zero and CFU counts on test days 7, 14, 21 and 28, one sample per wound per animal was collected on test day 0 for biofilm detection and all the animals exhibited biofilm presence on the wounds that were inoculated with Staphylococcus aureus inoculum with OD values on spectrophotometer. The wounds with inoculums showed mean OD value 0.299 (greater than 0.120 that indicates presence of biofilm). Similarly one sample per wound was collected on day 0 and necropsy days (Day 7, Day 14, Day 21 and Day 28), serially diluted from 10¹ to 10⁶ dilutions, plated and incubated. The 10⁶ dilution showed separate countable colonies and clear clones were considered for colony counting results evaluation. Results are shown in FIG. 7. The wounds treated with test composition 1 (test composition comprising 2% fusidic acid with chitosan biopolymer, closed circles in FIG. 7) and with test composition 3 (fusidic acid cream, closed triangles) showed least colony forming units on day 7 and day 14 as compared to wounds treated with test composition 2 (chitosan cream, closed squares) and with placebo (test composition 4, open diamonds). There were no colony forming units on test day 21 and test day 28 in all the wounds treated with any of the four compositions.

Histopathological evaluation of the wounds in the animals was done by processing wounds to a section thickness of 3-5 microns (for Haematoxylin and Eosin (H&E) and Sirius Red) and 2-3 microns (for immunohistochemistry (IHC)). The 3 to 5 microns wound tissue sections were stained by H & E and Sirius Red stains. Then the slides were examined under the light microscope by a study pathologist for evaluation of wound healing. The Sirius Red stained slides were analyzed for percent collagen proportion area (% CPA). The 2-3 microns sections were subjected to IHC staining to detect CD31 and VEGF markers and scored for positively stained area using the scoring system in Table 10-3. Results are shown in FIGS. 8A-8E, FIGS. 9A-9E, FIGS. 10A-10E, FIGS. 11A-11D and FIGS. 12A-12D.

TABLE 10-3
CD31 and VEGF Scoring of Wound Sites

| Grade | Severity |
|---|---|
| 1 | Minimal (Marker occupying less than 10% |
| | of tissue section) |
| 2 | Mild (Marker occupying 11 to 30% of tissue section) |
| 3 | Moderate (Marker occupying 31 to 60% of tissue section) |
| 4 | Severe/Marked (Marker occupying more than 60% of |
| | tissue section) |

### Example 11

### Preparation of Exemplary Gel Composition

A composition comprising fusidic acid was prepared with the components and in the amounts indicated in Table 11-1. Fusidic acid was formed in situ in the composition via reaction of sodium fusidate and nitric acid. Chitosan lactate was formed in situ in the composition via reaction of chitosan and lactic acid. The composition was transparent and a semi-solid, e.g., a gel, suitable for topical application to skin.

TABLE 11-1
Composition with Fusidic Acid

| | Amount | Category/ |
|---|---|---|
| Ingredients | % w/w | Role of Ingredient |
| Sodium fusidate (equivalent | 2.08 | Anti-bacterial |
| to form 2% fusidic acid) | | |
| Chitosan | 1.25 | Wound Healing/gelling agent |
| Edetate disodium | 0.10 | Chelating agent |
| Lactic acid¹ | 1.25 | Pharmaceutical aid |
| White petrolatum | 7.50 | Pharmaceutical aid |
| Cetostearyl alcohol | 7.50 | Emulsifying agent |
| Glycerin | 7.00 | Moisturizing agent |
| Polyoxyl 20 cetostearyl ether | 2.0 | Emulsifying agent |
| Polyethylene glycol (40) | 2.0 | Solubilizer/emulsifying agent |
| hydrogenated castor oil | | |
| (polyoxyl 40 hydrogenated | | |
| castor oil) | | |
| Collagen (marine hydrolyzed) | 0.20 | Moisturizing agent/ |
| | | Pharmaceutical aid |
| Light mineral oil | 5.00 | Emollient |
| Butylated hydroxytoluene | 0.01 | Antioxidant |
| Propylene glycol | 16.0 | Co-solvent |
| Nitric acid² | 0.392 | Pharmaceutical aid, for in situ |
| | | conversion of sodium fusidate to |
| | | fusidic acid |
| Dibasic sodium phosphate | 0.05 | Buffering agent |
| Benzoic acid | 0.20 | Anti-microbial preservative |
| Purified water | q.s. | Solvent |

¹Added as a processing aid for the formation of chitosan lactate from chitosan.
²Added as a processing aid for the formation of fusidic acid from sodium fusidate.

The gel composition of Table 11-1 was prepared as follows. The nitric acid was added to water to form a 1.605 molar solution. Separately, propylene glycol was heated with stirring to 67° C.±2° C.; the butylated hydroxytoluene was added, followed by polyoxyl 20 cetostearyl ether and polyoxyl 40 hydrogenated castor oil. The mixture was cooled to below 45° C. and sodium fusidate was added under nitrogen and with stirring until it was dissolved. The aqueous nitric acid solution was added under nitrogen to form a clear drug solution.

In a mixing vessel, edetate disodium and marine hydrolyzed collagen were added to water and dissolved. Lactic acid was then added, with stirring. Then chitosan was added with stirring until a clear gel was formed.

In another vessel, a water phase was prepared by adding to water 45° C.±2° C. dibasic sodium phosphate, polyoxyl 40 hydrogenated castor oil and glycerin. The water phase was heated to 72° C.±2° C. with continuous stirring.

In another vessel, an oil phase was prepared by mixing together the cetostearyl alcohol, white petrolatum, light mineral oil, polyoxyl 20 cetostearyl ether and benzoic acid to 72° C.±2° C. with continuous stirring.

The water phase and the oil phase were combined in a vessel under vacuum and homogenized, and the temperature of the vessel was cooled to 52° C.±1° C. by circulating cooling water through a jacket on the vessel. The drug solution was added, then the chitosan gel was added, both with mixing. The mixture was cooled to about 25° C.-30° C. with stirring to form the final gel composition.

### Example 12

### Method of Treating Wounds

Wound healing of four compositions including placebo was evaluated in a diabetic porcine full thickness wound model. The test compositions included (1) the 2% fusidic acid gel with chitosan biopolymer (Example 11), (2) chitosan gel, (3) fusidic acid gel, and (4) a placebo gel. Table 12-1 provides details of the compositions.

TABLE 12-1
Compositions for Wound Healing

| | | | Compo- |
|---|---|---|---|
| Composition | | Composition | sition |
| Identification | Active Ingredients | Appearance | pH |
| 12-1 | sodium fusidate, chitosan, nitric | gel | 3.71 |
| | oxide and lactic acid (Example 11) | | |
| 12-2 | chitosan, lactic acid | gel | 3.69 |
| 12-3 | sodium fusidate, nitric acid | gel | 3.77 |
| 12-4 | None (placebo) | gel | 3.98 |

Diabetes was created in test animals (females, 40.7 kg and 45.2 kg) by streptozotocin induction on day 1. After confirmation of a diabetic condition from glucose values, at day 15 total 24 full thickness wounds (2.5×2.5 cm²˜6-10 mm deep) were created on the animals and the four test compositions were tested (n=6). The treated wounds were covered with sterile non-adherent dressing, followed by combi-roll, brown gauze, vet wrap and elasticon. The animals were monitored for changes in wound area, glucose levels, and histopathological parameters. Only one of the two test animals complete the study to day 43, as one animal succumbed to death on day 26 for reasons unrelated to the test compositions. The animal that completed the study to day 43 exhibited improved wound healing with no adverse effects on behavior or health condition.

On days 18, 22, and 26 for test animal 1 and on days 18, 22, 29, 36 and 43 for test animal 2, the dressings were removed from the wounds for inspection by wound score, wound measurement, planimetry (L1× B1=A1, L2× B2=A2, Mean Area=A1+A2/2) and photography. Scoring systems are set forth in Tables 9-2 and 9-3. The wounds were treated again with the test compound and dressed. Wound healing was assessed by planimetry and quantitative assessment of wound scarring with photography on 18, 22 and 26 days (criteria and scoring according to Table 9-2).

In the test animal that completed the study to Day 26, Composition 12-1 showed mean area decreased 6.18±0.04 cm² on Day 18 to 4.42±0.78 cm² on Day 26. For Composition 12-2 this decreased from 6.18±0.04 cm² on Day 18 to 4.79±0.70 cm² on Day 26. For Composition 12-3 the mean area decreased from 6.15±0.05 cm² on Day 18 to 4.93±0.94 cm² on Day 26. For Composition 12-4 the mean area decreased from 6.18±0.04 cm² on Day 18 to 4.16±0.65 cm² on Day 26.

In the test animal that completed the study to Day 43, wound healing was assessed by planimetry and quantitative assessment of wound scarring with photography on 18, 22, 29, 36 and 43 days (criteria and scoring according to Table 9-2). Composition 12-1 showed mean area decreased 6.23±0.05 cm² on Day 18 to 1.16±0.45 cm² on Day 43. Composition 12-2 had a decrease from 6.25±0.00 cm² on Day 18 to 1.23±0.73 cm² on Day 43. For Composition 12-3 mean area decreased from 6.25±0.00 cm² on Day 18 to 1.57±0.43 cm² on Day 43. For Composition 12-4 this mean area decreased from 6.25±0.00 cm² on Day 18 to 1.61±0.65 cm² on Day 43.

In the quantitative assessment of wound scarring, the wounds treated with Composition 12-1 exhibited a noticeable gross mismatch compared to the surrounding skin on Day 18, which improved to a perfect color match by the necropsy day. The surface texture changed from a shiny appearance to a matte one, with mild to moderate distortion. Over the course of the study, the texture of these wounds transitioned from firm on Day 18 to normal by Day 43. A similar pattern was observed in wounds treated with Composition 12-2. Conversely, wounds treated with Composition 12-4 showed an obvious color mismatch on Day 18, which reduced to a slight mismatch by the necropsy day. The surface texture shifted from a slightly shiny appearance to matte, with mild to moderate distortion. During the study, the texture changed from firm on Day 18 to just palpable or normal by Day 43. A comparable pattern in wound scarring scores was observed in wounds treated with Composition 12-3.

In the test animal that completed the study to Day 26, the mean epithelialization (neo-epithelialization) and restoration of rete ridges score (epidermis) of wound treated with Compositions 12-1, 12-2, 12-3 and 12-4 were, respectively, 1.00 in all the treatments and 2.00 in all the treatments (criteria and scoring according to Table 9-3). The mean total score of collagen fiber orientation, density and maturity (Dermis) of wound treated with Compositions 12-1, 12-2, 12-3 and 12-4 were 9.00, 8.67, 8.67 and 9.50, respectively. The mean neovascularization score (dermis) of wound treated with Compositions 12-1, 12-2, 12-3 and 12-4 were 1.33, 1.33, 3.33 and 3.00, respectively. The mean inflammation score (dermis) of wound treated with Compositions 12-1, 12-2, 12-3 and 12-4 were 3.00, 2.83, 2.33 and 2.83, respectively. The mean scab score (dermis) of wound treated with Compositions 12-1, 12-2, 12-3 and 12-4 were 2.50, 2.83, 2.00 and 2.50, respectively.

In the test animal that completed the study to Day 43, the mean epithelialization (neo-epithelialization) score (epidermis) of wounds treated with Compositions 12-1, 12-2, 12-3 and 12-4 were 1.83, 2.00, 1.33 and 1.17, respectively (criteria and scoring according to Table 9-3). The mean restoration of rete ridges score (epidermis) of wound treated with Compositions 12-1, 12-2, 12-3 and 12-4 were 1.8 in all the treatments. The mean total score of collagen fiber orientation, density and maturity (dermis) of wound t treated with Compositions 12-1, 12-2, 12-3 and 12-4 were 6.67, 6.00, 4.50 and 7.33, respectively. The mean neovascularization score (dermis) of wounds treated with Compositions 12-1, 12-2, 12-3 and 12-4 were 2.50, 2.33, 3.00 and 3.67, respectively. The mean inflammation score (dermis) of wounds treated with Compositions 12-1, 12-2, 12-3 and 12-4 were 0.50, 0.50, 0.67 and 1.33, respectively. The mean scab score (dermis) of wound treated with Compositions 12-1, 12-2, 12-3 and 12-4 were 0.00, 0.17, 0.00 and 0.33, respectively.

### Example 13

Treatment of Epidermolysis Bullosa (EB) with Composition

A composition comprising fusidic acid is prepared as described in Example 11. Approximately twenty patients with any type of EB (e.g., documented diagnosis of simplex (EBSO, junctional (JEB—moderate and severe), DEB (moderate and severe subtypes)), aged ≥18 years old, are enrolled for a double-blind, vehicle controlled study to assess safety and efficacy of the composition. Each patient will have two comparable target wounds selected at screening by the investigator to treat each with the fusidic acid composition or with placebo vehicle (no fusidic acid and no chitosan), to allow for intra-patient comparison. Treatment with the fusidic acid composition or vehicle of each patient's target wound will be assigned by randomization. The fusidic acid composition or vehicle will be applied topically on the target wound at each dressing change with minimum frequency of every third day and maximum of once daily, for a period of 8 weeks. Patients record in a diary dressing changes and application of the composition to target wounds.

Screening procedures are done from Day-14 to Day-1 and baseline procedures are done on Day 1. Each patient returns to the study site for study assessments at study weeks 2, 4, 6 and 8. Standardized and validated digital photographic methods are utilized to measure wound area at each visit and to assess document healing. Safety and tolerability will be assessed by adverse events, local tolerability, vital signs, and physical examination throughout the study. Treatment of the EB wounds with the composition shows improved healing compared to wounds treated with placebo vehicle.

### Example 14

Treatment of EB with Composition

A composition comprising fusidic acid is prepared as described in Example 11. Subjects of both sexes with a diagnosis of epidermolysis bullosa (simplex, recessive dystrophic or non-Herlitz junctional), age over one year, and presence of two or more target lesions ranging in size from 5 to 250 cm² are enrolled for treatment. The composition of Example 11 is administered topically to the subjects for a period of 3 months with follow-up visits at 2, 4, 6, 8, 10 and 12 weeks after initiation of treatment.

A primary end point of the study is the size reduction of the target lesions or the closure measured by imaging the lesions. Secondary end points include parameters such as reduction or removal of skin lesion infection, assessment of the extent of skin involved in the lesion in percentage terms of total body surface area at month 3, pain assessment using the accepted clinical scales, and assessment of quality of life by completing the disease-specific questionnaires.

Treatment of the subjects shows lesion resolution within 60 days or a reduction in lesion size, with respect to baseline, of at least about 25% 30 days after treatment.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

It is claimed:

1. A composition, comprising:
fusidic acid or a salt thereof;
a biopolymer in an amount of greater than 1% w/w and less than about 5% w/w;
one or more hydrophilic solvents selected from glycerin, propylene glycol and both glycerin and propylene glycol,
a vegetable oil; and
collagen.

2. The composition of claim 1, wherein fusidic acid or a salt thereof is fusidic acid.

3. The composition of claim 1, wherein the biopolymer is chitosan.

4. The composition of claim 1, wherein the vegetable oil is a hydrogenated vegetable oil.

5. The composition of claim 4, wherein the hydrogenated vegetable oil is polyoxyl 40 hydrogenated castor oil.

6. The composition of claim 5, wherein the polyoxyl 40 hydrogenated castor oil is present in the composition in an amount of between about 0.5-5.0% w/w.

7. The composition of claim 1, wherein the collagen is marine collagen or hydrolyzed marine collagen.

8. The composition of claim 7, wherein the hydrolyzed marine collagen is present in the composition in an amount of between about 0.05-0.8% w/w.

9. The composition of claim 1, further comprising an oil mixture.

10. The composition of claim 9, wherein the oil mixture is present in the composition in an amount between about 8-15% w/w.

11. The composition of claim 9, wherein the oil mixture comprises a mineral oil and white petrolatum.

12. The composition of claim 11, wherein the mineral oil to white petrolatum are present in the oil mixture in a ratio of between 1:1 to 0.5:1.

13. The composition of claim 9, comprising between about 2.0-8.0% w/w mineral oil.

14. The composition of claim 1, further comprising an emulsifier comprised of a fatty alcohol and an ethoxylated fatty alcohol.

15. The composition of claim 14, wherein the fatty alcohol is cetostearyl alcohol, polyoxyl 20 cetostearyl ether, or a mixture of cetostearyl alcohol and polyoxyl 20 cetostearyl ether.

16. The composition of claim 14, wherein the fatty alcohol is cetostearyl alcohol and the composition comprises between about 5-9% w/w cetostearyl alcohol.

17. The composition of claim 14, wherein the fatty alcohol is polyoxyl 20 cetostearyl ether and the amount of polyoxyl 20 cetostearyl ether in the composition is between about 0.5-5.0% w/w.

18. A composition, comprising:
2% w/w fusidic acid,
1.25% w/w chitosan,
7% w/w glycerin,
16% w/w propylene glycol,
2% w/w polyoxyl 40 hydrogenated castor oil,
5% w/w light mineral oil,
7.5% w/w white petrolatum,
0.2% w/w marine hydrolyzed collagen,
2% w/w polyoxyl 20 cetostearyl ether, and
7.5% cetostearyl alcohol.

19. The composition of claim 18, further comprises one or more of water, lactic acid, benzoic acid, butylated hydroxytoluene, edetate disodium, and/or dibasic sodium phosphate.

20. The composition of claim 18, wherein the composition is translucent and/or semi-solid.

21. A method for treating skin in a person suffering from epidermolysis bullosa (EB), comprising:
providing a composition comprising fusidic acid and one or more pharmaceutically acceptable excipients; and
topically applying, or instructing to topically apply, the composition to skin of a person suffering from EB.

22. A method for treating a non-healing wound, a chronic wound or lesional skin in a person suffering from EB, comprising:
providing a composition comprising fusidic acid and one or more pharmaceutically acceptable excipients; and
topically applying, or instructing to topically apply, the composition to skin of a person suffering from EB.

23. A method for reducing likelihood of skin infection in a person suffering from EB, comprising:
providing a composition comprising fusidic acid and one or more pharmaceutically acceptable excipients; and
topically applying the composition to skin of a person suffering from EB.

24. A method for (i) treating skin dysbiosis or (ii) altering bacterial diversity of skin microbiome, comprising:
providing a composition comprising fusidic acid and one or more pharmaceutically acceptable excipients; and
topically applying the composition to skin of a person in need of treatment.

25. A method for improving quality of life in a subject suffering from EB, comprising:
providing a composition comprising fusidic acid and one or more pharmaceutically acceptable excipients; and
topically applying, or instructing to topically apply, the composition to skin of a subject suffering from EB,
whereby said topically applying improves quality of life as measured by one or more of a scale or scoring system selected from (i) a physician global assessment of pain, (ii) an investigator's global assessment of pain, (iii) a subject's global assessment of pain, and (iv) a measurement tool for itch intensity.

26. A method for reducing likelihood of scar formation in skin of in a person suffering from epidermolysis bullosa (EB), comprising:
providing a composition comprising fusidic acid and one or more pharmaceutically acceptable excipients; and
topically applying, or instructing to topically apply, the composition to skin of a person suffering from EB.

27. A method for preventing or reducing risk of a skin blister from becoming infected or for treating intact blisters on skin of a person suffering from EB, comprising:
providing a composition comprising fusidic acid and one or more pharmaceutically acceptable excipients; and
topically applying, or instructing to topically apply, the composition to skin of a person suffering from EB.
